(19) United States
(12) Patent Application Publication
(10) Pub. No.: US 2021/0046082 A1
(43) Pub. Date: Feb. 18, 2021

(54) CRYSTALLINE FORMS OF QUINOLINE ANALOGS AND SALTS THEREOF, COMPOSITIONS, AND THEIR METHODS FOR USE

(71) Applicant: Senhwa Biosciences, Inc., New Taipei City (TW)

(72) Inventor: Hshiou-ting LIU, Milpitas, CA (US)

(21) Appl. No.: 16/993,834

(22) Filed: Aug. 14, 2020

Related U.S. Application Data
(60) Provisional application No. 62/946,765, filed on Dec. 11, 2019.
(60) Provisional application No. 62/886,633, filed on Aug. 14, 2019.

(51) Int. Cl. A61K 31/551; C07D 513/14; A61K 31/501; A61K 39/395; A61P 35/02

(52) U.S. Cl. CPC A61K 31/551; C07D 513/14; C07B 2200/13; A61K 39/3955; A61P 35/02; A61K 31/501

(57) ABSTRACT

The present invention includes crystalline forms of 2-(4-Methyl-[1,4]diazepan-1-yl)-5-oxo-5H-7-thia-1,11b-diaza-benzo[c]fluorene-6-carboxylic acid (5-methyl-pyrazin-2-ylmethyl)-amide (Compound I). Furthermore, the present invention provides compositions comprising the crystalline forms and therapeutic use of the crystalline forms and the compositions thereof.

### CROSS-REFERENCE TO RELATED APPLICATIONS

This application claims the priority benefit of U.S. Provisional Application No. 62/886,633, filed Aug. 14, 2019 and U.S. Provisional Application No. 62/946,765, filed Dec. 11, 2019, the disclosures of which are incorporated by reference herein in their entireties.

### FIELD OF THE DISCLOSURE

The present invention generally relates to crystalline forms of 2-(4-Methyl-[1,4]diazepan-1-yl)-5-oxo-5H-7-thia-1,11b-diaza-benzo[c]fluorene-6-carboxylic acid (5-methyl-pyrazin-2-ylmethyl)-amide (Compound I). Furthermore, the present invention provides compositions comprising the crystalline forms and therapeutic use of the crystalline forms and the compositions thereof, such as for treating cancer.

### BACKGROUND OF THE DISCLOSURE

A variety of tetracyclic quinolone compounds have been suggested to function by interacting with quadruplex-forming regions of nucleic acids and modulating ribosomal RNA transcription. See, for example, U.S. Pat. Nos. 7,928,100 and 8,853,234. Specifically, the tetracyclic quinolone compounds can stabilize the DNA G-quadruplexes (G4s) in cancer cells and thereby induce synthetic lethality in cancer cells. Since treatment of cells with G4-stabilizing agents can lead to the formation of DNA double strand breaks (DSBs), DSB formation induced by G4-stabilizing ligand/agent (such as the tetracyclic quinolones) treatment would be more pronounced in cells genetically deficient in, or chemically inhibited in, repair pathways including both non-homologous end joining (NHEJ) and homologous recombination repair (HRR). Furthermore, the tetracyclic quinolone compounds selectively inhibit rRNA synthesis by Pol I in the nucleolus, but do not inhibit mRNA synthesis by RNA Polymerase II (Pol II) and do not inhibit DNA replication or protein synthesis. It is suggested that targeting RNA polymerase I (Pol I) to activate p53 through the nucleolar stress pathway may results in selective activation of p53 in tumor cells. The p53 protein normally functions as a tumor suppressor by causing cancer cells to self-destruct. Activating p53 to kill cancer cells is a well validated anticancer strategy and many approaches are being employed to exploit this pathway. Selective activation of p53 in tumor cells would be an attractive method of treating, controlling, ameliorating tumor cells while not affecting normal healthy cells. The aforementioned tetracyclic quinolones are disclosed in U.S. Pat. Nos. 7,928,100 and 8,853,234, and the contents of this publication are herein incorporated by reference in their entirety for all intended purposes.

Crystalline forms, including polymorphs, of an active pharmaceutical ingredient can offers advantages in controlling important physiochemical qualities, such as stability, solubility, bioavailability, particle size, bulk density, flow properties, polymorphic content, and other properties. Different salts and polymorphs of 2-(4-Methyl-[1,4]diazepan-1-yl)-5-oxo-5H-7-thia-1,11b-diaza-benzo[c]fluorene-6-carboxylic acid (5-methyl-pyrazin-2-ylmethyl)-amide have been discovered as disclosed in U.S. Pat. No. 9,957,282, which is hereby incorporated by reference in its entirety for all intended purposes. However, existence of polymorphs are especially difficult to predict and predicting its physical properties is even more challenging. Discovery of new polymorphs, thus, requires extensive experimental efforts. This disclosure relates to the discovery of new polymorphs, which surprisingly have good stability, which is suitable for pharmaceutical use.

### SUMMARY OF THE DISCLOSURE

In one embodiment, the present invention provides a crystalline form of Compound I or a pharmaceutically acceptable salt, ester, and/or solvate thereof. In one embodiment, the crystalline form of Compound I is a free base of Compound I. In another embodiment, the crystalline form of Compound I is a salt

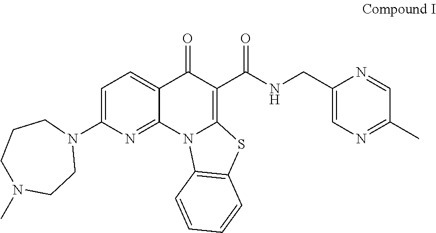 [SMILES: Cc1cnc(CNC(=O)c2c(=O)c3ccc(N4CCCN(C)CC4)nc3n3c2sc2ccccc23)cn1]

In one embodiment, the crystalline form of Compound I Polymorph J or Polymorph K.

In some embodiments, the crystalline form of the disclosure is isolated.

In some embodiments, the crystalline form is Compound I Polymorph J exhibiting X-ray powder diffraction (XRPD) pattern comprising peaks at about 5.5±0.5 and 11.0±0.5 degrees two-theta. In other embodiments, Compound I Polymorph J exhibits an XRPD pattern comprising peaks at about 7.1±0.5 degrees two-theta. In other embodiments, Compound I Polymorph J exhibits an XRPD pattern comprising peaks at about 17.7±0.5 and 26.7±0.5 degrees two-theta. In other embodiments, Compound I Polymorph J exhibits an XRPD pattern substantially similar to FIG. 1 or FIG. 2.

In some embodiments, Compound I Polymorph J exhibits a Differential Scanning Calorimetry (DSC) thermogram having a peak maximum between 200.0° C.±0.5° C. to 202.0±0.5° C. In other embodiments, Compound I Polymorph J exhibits a Differential Scanning Calorimetry (DSC) thermogram having a peak maximum between 238.0° C.±0.5° C. to 246.0±0.5° C.

In some embodiments, Compound I Polymorph J has a polymorphic purity of about 90% or higher. In other embodiments, Compound I Polymorph J has a polymorphic purity of about 95% or higher. In some embodiments, Compound I Polymorph J has a chemical purity of about 95% or higher. In some embodiments, Compound I Polymorph J has a chemical purity of about 98% or higher.

In some embodiments, the crystalline form is Compound I Polymorph K exhibiting an X-ray powder diffraction (XRPD) pattern comprising peaks at about 5.2±0.5 and 25.5±0.5 degrees two-theta. In other embodiments, Compound I Polymorph K exhibits an XRPD pattern comprising peaks at about 11.4±0.5 degrees two-theta. In other embodiments, Compound I Polymorph K exhibits an XRPD pattern comprising peaks at about 14.7±0.5 and 23.4±0.5 degrees two-theta. In other embodiments, Compound I Polymorph K exhibits an XRPD pattern substantially similar to FIG. 5.

In some embodiments, Compound I Polymorph K exhibits a Differential Scanning Calorimetry (DSC) thermogram having a peak maximum between 144.0° C. 0.5° C. to 150.0±0.5° C. In other embodiments, Compound I Polymorph K exhibits a DSC thermogram having a peak maximum between 231.0° C. 0.5° C. to 238.0±0.5° C. In other embodiments, Compound I Polymorph K exhibits a DSC thermogram having a peak maximum between 242.0° C.±0.5° C. to 250.0±0.5° C.

In some embodiments, Compound I Polymorph K has a polymorphic purity of about 90% or higher. In other embodiments, Compound I Polymorph K has a polymorphic purity of about 95% or higher. In some embodiments, Compound I Polymorph K has a chemical purity of about 95% or higher. In some embodiments, Compound I Polymorph K has a chemical purity of about 98% or higher.

In one embodiment, the disclosure relates to a composition comprising any one of the crystalline forms described herein. In some embodiments, the composition comprises at least one pharmaceutically acceptable carrier. In other embodiments, the composition further comprises one or more additional therapeutically active agent.

In one embodiment, the therapeutically active agent is selected from an alkylating agent, an anti-metabolite, a vinca alkaloid, a taxane, a topoisomerase inhibitor, an anti-tumor antibiotic, a tyrosine kinase inhibitor, an immunosuppressive macrolide, an Akt inhibitor, an HDAC inhibitor an Hsp90 inhibitor, an mTOR inhibitor, a PI3K/mTOR inhibitor, a PI3K inhibitor, a CDK (cyclin-dependent kinase) inhibitor, CHK (checkpoint kinase) inhibitor, or a PARP (poly (DP-ribose)polymerase) inhibitor. In one embodiment, the PARP inhibitor is olaparib.

In one embodiment, the therapeutically active agent is selected from an antibody or an antigen-binding portion thereof that disrupts the interaction between Programmed Death-1 (PD-1) and Programmed Death Ligand-1 (PD-L1). In other embodiments, the therapeutically active agent is selected from an anti-PD-1 antibody, a PD-1 antagonist, an anti-PD-L1 antibody, a siRNA targeting expression of PD-1, a siRNA targeting the expression of PD-L1, and a peptide, fragment, dominant negative form, or soluble form of PD-1 or PD-L1.

In one embodiment of the present disclosure, methods for stabilizing G-quadruplexes (G4s) in a subject are provided, wherein the method comprises administering to the subject a therapeutically effective amount of a crystalline form of the present disclosure or the composition comprising a therapeutically effective amount of a crystalline form of the present disclosure. In one embodiment, the stabilizing G4s is in peripheral blood mononuclear cells.

In one embodiment of the present disclosure, methods for modulating p53 activity in a subject are provided, wherein the method comprising administering to the subject a therapeutically effective amount of a crystalline form of the present disclosure or the composition comprising a therapeutically effective amount of a crystalline form of the present disclosure.

In one embodiment of the present disclosure, methods for treating or ameliorating cell proliferation disorder in a subject are provided, wherein the method comprising administering to a subject in need thereof a therapeutically effective amount of a crystalline form of the present disclosure or the composition comprising a therapeutically effective amount of a crystalline form of the present disclosure.

In some embodiments of any one of the methods disclosed herein, the cell proliferation disorder is cancer. In some embodiments, the cancer is selected from hematologic malignancy, colorectal cancer, breast cancer, lung cancer, liver cancer, ovarian cancer, cervical cancer, Ewing's sarcoma, pancreatic cancer, cancer of the lymph nodes, colon cancer, prostate cancer, brain cancer, bone cancer, cancer of the head and neck, skin cancer, kidney cancer, osteosarcoma, cancer of the heart, uterine cancer, gastrointestinal malignancies, and carcinomas of the larynx and oral cavity. In one embodiment, the cancer is breast cancer, ovarian cancer, or pancreatic cancer.

In one embodiment of any one of the methods disclosed herein, the cancer is hematologic malignancy. In one embodiment, the cancer is selected from leukemia, lymphoma, myeloma, and multiple myeloma.

In one embodiment of any one of the methods disclosed herein, the cell proliferation disorder is a solid tumor.

In one embodiment of any one of the methods disclosed herein, the cancer is a BRCA mutant or BRCA-like mutant cancer. In one embodiment, the cancer is a BRCA mutant cancer. In some embodiments, the cancer is a BRCA2-mutated cancer. In one embodiment, the BRCA mutant or BRCA-like mutant cancer is breast cancer, ovarian cancer, pancreatic cancer, or prostate cancer. In one embodiment, the BRCA mutant cancer is breast cancer, ovarian cancer, pancreatic cancer, or prostate cancer. In one embodiment, the BRCA mutant cancer is breast cancer. In another embodiment, the BRCA mutant cancer is ovarian cancer

In one embodiment of any one of the methods disclosed herein, the cancer is BRCA2 deficient or BRCA1 deficient cancer. In one embodiment, the cancer is BRCA2 deficient cancer.

In some embodiments of any one of the methods disclosed herein, the subject has a mutation in a DNA repair gene. In one embodiment, the DNA repair gene is a gene in the homologous recombination (HR) or non-homologous end joining (NHEJ) dependent deoxyribonucleic acid (DNA) double strand break (DSB) repair pathway.

### BRIEF DESCRIPTION OF THE DRAWINGS

FIG. 1 is a graph of an x-ray powder diffraction (XRPD) pattern of Polymorph J of Compound I (free base).

FIG. 2 is a graph of an x-ray powder diffraction (XRPD) pattern of Polymorph J of Compound I (free base).

FIG. 3 is a differential scanning calorimetry (DSC) thermogram of two different samples of Polymorph J which corresponds to sample shown in FIGS. 1 and 2.

FIG. 4 is an overlay of thermogravimetric analysis (TGA) thermograms of Polymorphs A, E, J, and K of Compound I (free base).

FIG. 5 is an overlay of XRPD patterns of Polymorphs A, E, J, and K of Compound I (free base).

FIG. 6 is an overlay of DSC thermograms of Polymorphs A, E, J, and K of Compound I (free base).

FIG. 7 is an overlay of XRPD patterns of Polymorphs A and J, and 1:1 mixture of Polymorphs A and J heated in MeOH/PPW (3:1) at 65° C. as observed at T=0 hr, 2 hrs, 4 hrs, and 6 hrs.

FIG. 8 is an overlay of XRPD patterns of Polymorphs A and J, and Polymorph J heated in MeOH/PPW (3:1) at 65° C. after 6 hrs.

FIG. 9 is an overlay of XRPD patterns of Polymorphs A, E and J, with Polymorph J heated at 205° C. for 30 minutes.

FIG. 10 is an XRPD patterns of Polymorph J after 18 months at 25° C./60% RH.

FIG. 11 is a DSC thermogram of Polymorph J after 18 months at 25° C./60% RH.

FIG. 12 is an overlay of VT-XRPD pattern of Polymorph J after 18 months at 25° C./60% RH.

FIG. 13A is a Raman spectrum of Polymorph J. FIG. 13B is a Raman spectrum of Polymorph K. FIG. 13C is a Raman spectrum of Polymorph A. FIG. 13D is a Raman spectrum of Polymorph E.

FIG. 14 shows best % tumor shrinkage from baseline for evaluable patients with genetic mutations labelled.

FIG. 15 shows duration on therapy for all patients at each dose level with genetic mutations labelled.

FIG. 16 depicts CT scans of a patient who harbored a PALB2 mutation and showed partial response (PR) (dosed at 650 mg/m² on Day 1, Day 8, and Day 15 of a 28 day cycle), A) prior to treatment with Compound I and B) 6-month follow-up scan following treatment with Compound I.

### DETAILED DESCRIPTIONS OF THE DISCLOSURE

The present invention relates to crystalline forms of 2-(4-Methyl-[1,4]diazepan-1-yl)-5-oxo-5H-7-thia-1,11b-diaza-benzo[c]fluorene-6-carboxylic acid (5-methyl-pyrazin-2-ylmethyl)-amide (Compound I) or salts or solvates thereof. Compound I or salts or solvates thereof, including its crystalline forms, can stabilize G-quadruplexes (G4s) and/or inhibit Pol I. These crystalline materials can be useful for treating disorders characterized by proliferation of cells.

### Definitions

It is to be understood that the terminology used herein is for the purpose of describing particular embodiments only and is not intended to be limiting.

Unless defined otherwise, all technical and scientific terms used herein have the same meaning as commonly understood to one of ordinary skill in the art to which the present application belongs. Although any methods and materials similar or equivalent to those described herein can be used in the practice or testing of the present application, representative methods and materials are herein described.

Following long-standing patent law convention, the terms “a”, “an”, and “the” refer to “one or more” when used in this application, including the claims. Thus, for example, reference to “a carrier” includes mixtures of one or more carriers, two or more carriers, and the like.

The term “compound(s) of the present invention” or “present compound(s)” refers to crystalline forms of 2-(4-Methyl-[1,4]diazepan-1-yl)-5-oxo-5H-7-thia-1,11b-diaza-benzo[c]fluorene-6-carboxylic acid (5-methyl-pyrazin-2-ylmethyl)-amide (Compound I) or isomers, salts, N-oxides, sulfoxides, sulfones, or solvates thereof. The crystalline forms of Compound I described throughout the application including a crystalline form of any single isomer of Compound I, a mixture of any number of isomers of Compound I. The crystalline forms include polymorphs.

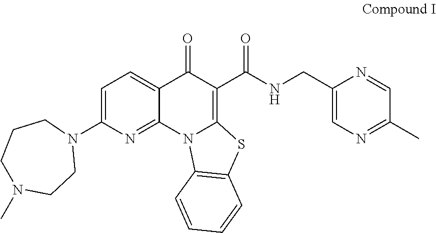 [SMILES: Cc1cnc(CNC(=O)c2c(=O)c3ccc(N4CCCN(C)CC4)nc3n3c2sc2ccccc23)cn1]

Polymorphism can be characterized as the ability of a compound to crystallize into different crystal forms, while maintaining the same chemical formula. Different polymorphs of the same compound (same chemical formula) exists in different crystalline phases that have different arrangements and/or conformation of the molecule in the crystal lattice. As used herein, a polymorph includes crystalline form of a compound (including Compound I) as well as its salts, solvates or hydrates. Polymorphism can affect one or more physical properties, such as stability, solubility, melting point, bulk density, flow properties, bioavailability, etc.

The term “impurity” of a compound, as used herein, means chemicals other than the compound, including, derivatives of the compound, or degradants of the compound that remain with the compound due to incomplete purification, or that develop over time, such as during stability testing, formulation development of the compound or storage of the compound.

The term “chemical purity” of a compound, as used herein, refers to the purity of a compound from other distinct chemical entities. For example, crystalline Compound I having 90% chemical purity means that the crystalline form contains less than 10% of molecules or chemical entity different from Compound I, including synthetic byproducts, residual solvents, or residual organic or inorganic substances.

The term “polymorphic purity” of a compound, as sued herein, refers to the purity of a compound to exist in one distinct polymorphic form. For example, Compound I Polymorph J having a polymorphic purity of 90% means that the crystalline form contains less than 10% of other polymorphic forms of Compound I in total, such as Polymorph A, C, E, G, and/or J. Polymorphs A, C, E, and G, are as disclosed in U.S. Pat. No. 9,957,282, which is hereby incorporated by reference in its entirety.

The term “isomer” refers to compounds having the same chemical formula but may have different stereochemical formula, structural formula, or special arrangements of atoms. Examples of isomers include stereoisomers, diastereomers, enantiomers, conformational isomers, rotamers, geometric isomers, and atropisomers.

“N-oxide”, also known as amine oxide or amine-N-oxide, means a compound that derives from a compound of the present invention via oxidation of an amine group of the compound of the present invention. An N-oxide typically contains the functional group R₃N⁺—O⁻ (sometimes written as R₃N═O or R₃N→O).

The term “ester” refers to any ester of a compound of the present invention in which any of the —COOH functions of the molecule is replaced by a —COOR function, in which the R moiety of the ester is any carbon-containing group which forms a stable ester moiety, including but not limited to alkyl, alkenyl, alkynyl, cycloalkyl, cycloalkylalkyl, aryl, arylalkyl, heterocyclyl, heterocyclylalkyl and substituted derivatives thereof. The term “ester” includes but is not limited to pharmaceutically acceptable esters thereof. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids.

“Sulfoxide” refers to a compound that derived from a compound of the present invention via oxidation of a sulfur (S) atom. Sulfoxides are commonly written as —S(═O)—, —S(O)—, or —(S→O)—. “Sulfone” refers to a compound that derived from a compound of the present invention via further oxidation of a sulfur atom. Sulfones are commonly written as —S(═O)₂—, —S(O)₂—, or —(S(→O)₂)—.

The term “carboxylic acid” refers to an organic acid characterized by one or more carboxyl groups, such as acetic acid and oxalic acid. “Sulfonic acid” refers to an organic acid with the general formula of R—(S(O)₂—OH)ₙ, wherein R is an organic moiety and n is an integer above zero, such as 1, 2, and 3. The term “polyhydroxy acid” refers to a carboxylic acid containing two or more hydroxyl groups. Examples of polyhydroxy acid include, but are not limited to, lactobionic acid, gluconic acid, and galactose.

The term “composition” denotes one or more substance in a physical form, such as solid, liquid, gas, or a mixture thereof. One example of composition is a pharmaceutical composition, i.e., a composition related to, prepared for, or used in medical treatment. The term “formulation” is also used to indicate one or more substance in a physical form, such as solid, liquid, gas, or a mixture thereof.

The term “co-administration” or “coadministration” refers to administration of a formulation or a composition comprising (a) a compound of the invention or a formulation prepared from a compound of the invention; and (b) one or more additional therapeutic agent and/or radio therapy, in combination, i.e., together in a coordinated fashion.

As used herein, “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

“Salts” include derivatives of an active agent, wherein the active agent is modified by making acid or base addition salts thereof. Preferably, the salts are pharmaceutically acceptable salts. Such salts include, but are not limited to, pharmaceutically acceptable acid addition salts, pharmaceutically acceptable base addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like. Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine dicyclohexylamine and the like. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like. Standard methods for the preparation of pharmaceutically acceptable salts and their formulations are well known in the art, and are disclosed in various references, including for example, “Remington: The Science and Practice of Pharmacy”, A. Gennaro, ed., 20th edition, Lippincott, Williams & Wilkins, Philadelphia, Pa.

As used herein, “solvate” means a complex formed by solvation (the combination of solvent molecules with molecules or ions of the compounds of the present invention), or an aggregate that consists of a solute ion or molecule (the compounds of the present invention) with one or more solvent molecules. In the present invention, the preferred solvate is hydrate. Examples of hydrate include, but are not limited to, hemihydrate, monohydrate, dihydrate, trihydrate, hexahydrate, etc. It should be understood by one of ordinary skill in the art that the pharmaceutically acceptable salt of the present compound may also exist in a solvate form. The solvate is typically formed via hydration which is either part of the preparation of the present compound or through natural absorption of moisture by the anhydrous compound of the present invention. Solvates including hydrates may be consisting in stoichiometric ratios, for example, with two, three, four salt molecules per solvate or per hydrate molecule. Another possibility, for example, that two salt molecules are stoichiometric related to three, five, seven solvent or hydrate molecules. Solvents used for crystallization, such as alcohols, especially methanol and ethanol; aldehydes; ketones, especially acetone; esters, e.g. ethyl acetate; may be embedded in the crystal grating. Preferred are pharmaceutically acceptable solvents.

The term “substantially similar” as used herein with regards to bioavailability of pharmacokinetics means that the two or more therapeutically active agents or drugs provide the same therapeutic effects in a subject.

The term “substantially similar” as used herein with regards to an analytical spectrum, such as XRPD patterns, Raman spectroscopy, etc., means that a spectrum resembles the reference spectrum to a great degree in both the peak locations and their intensity.

The term “substantially free of” as used herein, means free from therapeutically effective amounts of compounds when administered in suggested doses, but may include trace amounts of compounds in non-therapeutically effective amounts.

The terms “excipient”, “carrier”, and “vehicle” are used interchangeably throughout this application and denote a substance with which a compound of the present invention is administered.

“Therapeutically effective amount” means the amount of a therapeutically active agent, when administered to a patient for treating a disease or other undesirable medical condition, is sufficient to have a beneficial effect with respect to that disease or condition. The therapeutically effective amount will vary depending on the identity of the therapeutically active agent, the disease or condition and its severity, and the age, weight, etc. of the patient to be treated. Determining the therapeutically effective amount of the therapeutically active agent is within the ordinary skill of the art and requires no more than routine experimentation.

As used herein, the terms “additional pharmaceutical agent” or “additional therapeutic agent” or “additional therapeutically active agent” with respect to the compounds described herein refers to an active agent other than the Compound I, or a pharmaceutically acceptable salt, ester, or solvate thereof, which is administered to elicit a therapeutic effect. The pharmaceutical agent(s) may be directed to a therapeutic effect related to the condition that the compounds of the present disclosure is intended to treat or ameliorate (e.g., cancer) or, the pharmaceutical agent may be intended to treat or ameliorate a symptom of the underlying condition (e.g., tumor growth, hemorrhage, ulceration, pain, enlarged lymph nodes, cough, jaundice, swelling, weight loss, cachexia, sweating, anemia, paraneoplastic phenomena, thrombosis, etc.) or to further reduce the appearance or severity of side effects of the compounds of the present disclosure.

As used herein, the phrase “a disorder characterized by cell proliferation” or “a condition characterized by cell proliferation” include, but are not limited to, cancer, benign and malignant tumors. Examples of cancer and tumors include, but are not limited to, cancers or tumor growth of the large intestine, breast (including inflammatory breast cancer), lung, liver, pancreas, lymph node, colon, rectum, prostate, brain, head and neck, skin, kidney, osteosarcoma, blood and heart (e.g., leukemia, lymphoma, and carcinoma).

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

The term “patient” or “subject” as used herein, includes humans and animals, preferably mammals.

As used herein, the terms “inhibiting” or “reducing” cell proliferation is meant to slow down, to decrease, or, for example, to stop the amount of cell proliferation, as measured using methods known to those of ordinary skill in the art, by, for example, 10%, 20%, 30%, 40%, 50%, 60%, 70%, 80%, 90%, 95%, or 100%, when compared to proliferating cells that are not subjected to the methods and compositions of the present application.

As used herein, the term “apoptosis” refers to an intrinsic cell self-destruction or suicide program. In response to a triggering stimulus, cells undergo a cascade of events including cell shrinkage, blebbing of cell membranes and chromatic condensation and fragmentation. These events culminate in cell conversion to clusters of membrane-bound particles (apoptotic bodies), which are thereafter engulfed by macrophages.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the present application.

### Compound I

Compound I is a synthetically derived small molecule, which can selectively binds and stabilizes DNA G-quadruplex (G4) structures. Key attributes of Compound I include induction of DNA damage through G4 stabilization which is dependent on intact BRCA and other homologous recombination mediated pathways for resolution. Cumulative mutations affecting BRCA and homologous recombination (HR) deficient tumor cells result in synthetic lethality.

Compound I showed specific toxicity against BRCA deficient cells in a number of cell lines of different genetic backgrounds (colon, breast and ovary) and different specifies origin (mouse and human). Compound I exhibited a wide therapeutic index of activity in BRCA2 knockout tumor cells in a xenograft model, when compared with isogenic wild type control cells. Without bound to any theory, the data to date attribute the anti-tumor activity of Compound I to bind and stabilize G4 DNA structure and impede the progression of DNA replication complexes and induces single stranded DNA gaps or breaks. The BRCA pathway is required for the repair of Compound I induced DNA damage, and that compromised DNA damage repair in the absence of BRCA genes will lead to lethality. BRCA deficient cells can be killed by Compound I at low drug concentration which are not effective at inhibiting rDNA transcription which suggests, without bound to any theory, that the dose used to treat BRCA deficient cancers is lower than that required to inhibit RNA Polymerase I and disrupt nucleons function.

Further, Compound I has shown to be responsive to PALB2 mutated cancers. The PALB2 gene is called the partner and localizer of the BRCA2 gene. It provides instructions to make a protein that works with the BRCA2 protein to repair damaged DNA and stop tumor growth. Inheriting two abnormal PALB2 genes causes Fanconi anemia type N, which suppresses bone marrow function and leads to extremely low levels of red blood cells, white blood cells, and platelets.

Compound I exhibited antiproliferation activity against a variety of cancer cell lines. See Example 6.

### Crystalline Materials

In one embodiment, the present disclosure provides a crystalline form of Compound I (free base). In another embodiment, the present invention provides a crystalline form of a salt and/or solvate of Compound I. In one embodiment, the present disclosure provides an isolated crystalline form of Compound I or a salt, ester, or a solvate thereof.

In one embodiment, the salt is a hydrochloric acid addition salt, a sulfuric acid addition salt, a sulfonic acid addition salt, a carboxylic acid addition salt, or a polyhydroxy acid addition salt. Examples of the crystalline salt include, but are not limited to, hydrochloric acid salt, maleic acid salt, fumaric acid salt, citric acid salt, malic acid salt, sulfuric acid salt, acetic acid salt, phosphoric acid salt, L-(+)-tartaric acid salt, D-glucuronic acid salt, benzoic acid salt, succinic acid salt, ethane sulfonic acid salt, methane sulfonic acid salt, p-toluene sulfonic acid salt, malonic acid salt, benzene sulfonic acid salt, and 1-hydroxy-2-naphthoic acid salt.

In one embodiment, the crystalline forms are characterized by the interlattice plane intervals determined by an X-ray powder diffraction (XRPD) pattern. The spectrum of XRPD is typically represented by a diagram plotting the intensity of the peaks versus the location of the peaks, i.e., diffraction angle 20 (two-theta) in degrees. The intensities are often given in parenthesis with the following abbreviations: very strong=vst; strong=st; medium=m; weak=w; and very weak=vw. The characteristic peaks of a given XRPD can be selected according to the peak locations and their relative intensity to conveniently distinguish this crystalline structure from others. The % intensity of the peaks relative to the most intense peak may be represented as I/Io.

Those skilled in the art recognize that the measurements of the XRPD peak locations and/or intensity for a given crystalline form of the same compound will vary within a margin of error. The values of degree 20 allow appropriate error margins. Typically, the error margins are represented by “±”. For example, the degree 20 of about “8.7±0.3” denotes a range from about 8.7±0.3, i.e., about 9.0, to about 8.7-0.3, i.e., about 8.4. Depending on the sample preparation techniques, the calibration techniques applied to the instruments, human operational variation, and etc., those skilled in the art recognize that the appropriate error of margins for a XRPD can be about ±1.0; ±0.9; ±0.8; ±0.7; ±0.6; ±0.5; ±0.4; ±0.3; ±0.2; ±0.1; ±0.05; or less.

Additional details of the methods and equipment used for the XRPD analysis are described in the Examples section.

In one embodiment, the crystalline forms are characterized by Differential Scanning Calorimetry (DSC). The DSC thermogram is typically expressed by a diagram plotting the normalized heat flow in units of Watts/gram (“W/g”) versus the measured sample temperature in degree C. The DSC thermogram is usually evaluated for extrapolated onset and end (outset) temperatures, peak temperature, and heat of fusion. A peak characteristic value of a DSC thermogram is often used as the characteristic peak to distinguish this crystalline structure from others.

Those skilled in the art recognize that the measurements of the DSC thermogram for a given crystalline form of the same compound will vary within a margin of error. The values of a single peak characteristic value, expressed in degree C., allow appropriate error margins. Typically, the error margins are represented by “±”. For example, the single peak characteristic value of about “53.09±2.0” denotes a range from about 53.09±2.0, i.e., about 55.09, to about 53.09-2.0, i.e., about 51.09. Depending on the sample preparation techniques, the calibration techniques applied to the instruments, human operational variations, and etc., those skilled in the art recognize that the appropriate error of margins for a single peak characteristic value can be ±2.5; ±2.0; ±1.5; ±1.0; ±0.5; or less.

Additional details of the methods and equipment used for the DSC thermogram analysis are described in the Examples section.

In one embodiment, the crystalline forms are characterized by Raman spectroscopy. The Raman spectrum is typically represented by a diagram plotting the Raman intensity of the peaks versus the Raman shift of the peaks. The “peaks” of Raman spectroscopy are also known as “absorption bands”. The intensities are often given in parenthesis with the following abbreviations: strong=st; medium=m; and weak=w. The characteristic peaks of a given Raman spectrum can be selected according to the peak locations and their relative intensity to conveniently distinguish this crystalline structure from others.

Those skilled in the art recognize that the measurements of the Raman peak shifts and/or intensity for a given crystalline form of the same compound will vary within a margin of error. The values of peak shift, expressed in reciprocal wave numbers (cm⁻¹), allow appropriate error margins. Typically, the error margins are represented by “±”. For example, the Raman shift of about “1310±10” denotes a range from about 1310±10, i.e., about 1320, to about 1310-10, i.e., about 1300. Depending on the sample preparation techniques, the calibration techniques applied to the instruments, human operational variations, and etc., those skilled in the art recognize that the appropriate error of margins for a Raman shift can be ±12; ±10; ±8; ±5; ±3; ±1; or less.

Additional details of the methods and equipment used for the Raman spectroscopy analysis are described in the Examples section.

In one embodiment, the compounds of the present invention is a free base (Compound I free base). In one embodiment, the compounds of the present invention is selected from Polymorph J or Polymorph K.

In some embodiments, the compound of the present invention has a chemical purity greater than about 50%, about 60%, about 70%, about 80%, about 85%, about 95%, about 98%, or any values in between (i.e., greater than about 83%, greater than about 97%, etc.). In some embodiments, the compound of the present invention has a chemical purity greater than about 80%, about 81%, about 82%, about 83%, about 84%, about 85%, about 86%, about 87%, about 88%, about 89%, about 90%, about 91%, about 92%, about 93%, about 94%, or about 95%. In some embodiments, the compound of the present invention has a chemical purity greater than about 90%. In some embodiments, the compound of the present invention has a chemical purity greater than about 95%. In some embodiments, the compound of the present invention has a chemical purity greater than about 98%. In some embodiments, the compound of the present invention has a chemical purity greater than about 99%.

In some embodiments, the compound of the present invention has a polymorphic purity greater than about 50%, about 55%, about 60%, about 65%, about 70%, about 75% about 80%, about 85%, about 90%, about 95%, about 98%, or any values in between. In some embodiments, the compound of the present invention has a polymorphic purity greater than about 80%, about 81%, about 82%, about 83%, about 84%, about 85%, about 86%, about 87%, about 88%, about 89%, about 90%, about 91%, about 92%, about 93%, about 94%, or about 95%. In some embodiments, the compound of the present invention has a polymorphic purity greater than about 90%. In some embodiments, the compound of the present invention has a polymorphic purity greater than about 95%. In some embodiments, the compound of the present invention has a polymorphic purity greater than about 98%. In some embodiments, the compound of the present invention has a polymorphic purity greater than about 99%.

In some embodiments, Compound I Polymorph J has a polymorphic purity greater than 80%. In some embodiments, Compound I Polymorph J has a polymorphic purity greater than 85%. In some embodiments, Compound I Polymorph J has a polymorphic purity greater than 90%. In some embodiments, Compound I Polymorph J has a polymorphic purity greater than 95%. In some embodiments, Compound I Polymorph J has a polymorphic purity greater than 98%. In some embodiments, Compound I Polymorph J is isolated.

In some embodiments, Compound I Polymorph K has a polymorphic purity greater than 80%. In some embodiments, Compound I Polymorph K has a polymorphic purity greater than 85%.In some embodiments, Compound I Polymorph K has a polymorphic purity greater than 90%. In some embodiments, Compound I Polymorph K has a polymorphic purity greater than 95%. In some embodiments, Compound I Polymorph K has a polymorphic purity greater than 98%. In some embodiments, Compound I Polymorph K is isolated.

In one embodiment of the present disclosure, the crystalline form of Compound I may comprise of a mixture of Compound I Polymorph J and one or more other forms of polymorphs of Compound I, including Polymorphs A, C, E, and G as disclosed in U.S. Pat. No. 9,957,282, which is hereby incorporated by reference in its entirety, or Polymorph K. In some embodiments, the crystalline form of Compound I may comprise of substantially pure form of Polymorph J. In one embodiment, the crystalline form of Compound I may comprise of over about 99.9%, about 99.8%, about 99.7%, about 99.6%, about 99.5%, about 99.4%, about 99.3%, about 99.2%, about 99.1%, or about 99.0% of Compound I Polymorph J. In another embodiment, the crystalline form of Compound I may comprise over about 99%, 98%, 97%, 96%, 95%, 94%, 93%, 92%, 91%, or 90% of Compound I Polymorph J. In some embodiments, the crystalline form of Compound I may comprise over about 90%, 85%, 80%, 75%, 70%, 65%, 60%, 55%, 50%, 45%, or 40% of Compound I Polymorph J.

In one embodiment of the present disclosure, the crystalline form of Compound I may comprise of a mixture of Compound I Polymorph K and one or more other forms of polymorphs of Compound I, including Polymorphs A, C, E, and G as disclosed in U.S. Pat. No. 9,957,282, which is hereby incorporated by reference in its entirety, or Polymorph J. In some embodiments, the crystalline form of Compound I may comprise of substantially pure form of Polymorph K. In one embodiment, the crystalline form of Compound I may comprise of over about 99.9%, about 99.8%, about 99.7%, about 99.6%, about 99.5%, about 99.4%, about 99.3%, about 99.2%, about 99.1%, or about 99.0% of Compound I Polymorph K. In another embodiment, the crystalline form of Compound I may comprise over about 99%, 98%, 97%, 96%, 95%, 94%, 93%, 92%, 91%, or 90% of Compound I Polymorph K. In some embodiments, the crystalline form of Compound I may comprise over about 90%, 85%, 80%, 75%, 70%, 65%, 60%, 55%, 50%, 45%, or 40% of Compound I Polymorph K.

In some embodiments, the compound of the present invention comprises Compound I Polymorph J in combination with other polymorphs of Compound I. In some embodiments, the compound of the present invention comprises Compound I Polymorph J and Polymorph A or Polymorph E. In some embodiments, the compound of the present invention comprises Compound I Polymorph J and Polymorph A. In some embodiments, the compound of the present invention comprises Compound I Polymorph J and Polymorph E. In some embodiments, the compound of the present invention comprises Compound I Polymorph J, Polymorph A, and Polymorph E.

In one embodiment of the present disclosure, the compound of the present invention comprises Compound I Polymorph J and Polymorph A in any amount or any combination. In one embodiment of the present disclosure, the compound of the present invention comprises Compound I Polymorph J and about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, or about 20% of Polymorph A. one embodiment of the present disclosure, the compound of the present invention comprises Compound I Polymorph J and about 20%, about 30%, about 40%, about 50%, about 60%, about 70%, about 80%, about 90%, about 95%, or any value in between, of Polymorph A.

In one embodiment of the present disclosure, the compound of the present invention comprises Compound I Polymorph J and Polymorph E in any amount or any combination. In one embodiment of the present disclosure, the compound of the present invention comprises Compound I Polymorph J and about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, or about 20% of Polymorph E. one embodiment of the present disclosure, the compound of the present invention comprises Compound I Polymorph J and about 20%, about 30%, about 40%, about 50%, about 60%, about 70%, about 80%, about 90%, about 95%, or any value in between, of Polymorph E.

In one embodiment of the present disclosure, the compound of the present invention comprises Compound I Polymorph K and Polymorph A in any amount or any combination. In one embodiment of the present disclosure, the compound of the present invention comprises Compound I Polymorph K and about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, or about 20% of Polymorph A. one embodiment of the present disclosure, the compound of the present invention comprises Compound I Polymorph K and about 20%, about 30%, about 40%, about 50%, about 60%, about 70%, about 80%, about 90%, about 95%, or any value in between, of Polymorph A.

In one embodiment of the present disclosure, the compound of the present invention comprises Compound I Polymorph K and Polymorph E in any amount or any combination. In one embodiment of the present disclosure, the compound of the present invention comprises Compound I Polymorph K and about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, or about 20% of Polymorph E. one embodiment of the present disclosure, the compound of the present invention comprises Compound I Polymorph K and about 20%, about 30%, about 40%, about 50%, about 60%, about 70%, about 80%, about 90%, about 95%, or any value in between, of Polymorph E.

In some embodiments, the compound of the present invention comprises Compound I Polymorph K in combination with other polymorphs of Compound I. In some embodiments, the compound of the present invention comprises Compound I Polymorph K and Polymorph A or Polymorph E. In some embodiments, the compound of the present invention comprises Compound I Polymorph K and Polymorph A. In some embodiments, the compound of the present invention comprises Compound I Polymorph K and Polymorph E. In some embodiments, the compound of the present invention comprises Compound I Polymorph K, Polymorph A, and Polymorph E.

In some embodiments, the compounds of the present invention is stable during storage. In some embodiments, the compounds of the present invention is stable at ambient temperature. In some embodiments, the compounds of the present invention is stable at ambient temperature for at least 6 months, at least 12 months, or at least 18 months. In some embodiments, substantially pure compounds of the present invention is stable at ambient temperature for at least 6 months, at least 12 months, or at least 18 months.

In some embodiments, the compounds of the present invention is stable at 25° C./60% RH. In some embodiments, the compounds of the present invention is stable at 25° C./60% RH for at least 6 months, at least 12 months, or at least 18 months. In some embodiments, substantially pure compounds of the present invention is stable at 25° C./60% RH for at least 6 months, at least 12 months, or at least 18 months.

In some embodiments, the compounds of the present invention which has a polymorphic purity of at least 90% is stable at ambient temperature for at least 6 months, at least 12 months, or at least 18 months. In some embodiments, the compounds of the present invention which has a polymorphic purity of at least 95% is stable at ambient temperature for at least 6 months, at least 12 months, or at least 18 months. In some embodiments, the compounds of the present invention which has a polymorphic purity of at least 98% is stable at ambient temperature for at least 6 months, at least 12 months, or at least 18 months. In some embodiments, isolated compounds of the present invention is stable at ambient temperature for at least 6 months, at least 12 months, or at least 18 months.

Polymorph A, as disclosed herein, is a polymorph of a free base of Compound I which exhibits an XRPD pattern comprising peaks at about 7.7, 22.1, and 24.6 degrees two-theta with the margin of error of about ±0.5; about ±0.4; about ±0.3; about ±0.2; or about ±0.1; or less. In another embodiment, the XRPD of the crystalline Polymorph A further comprises peaks at about 9.4±0.5 and 27.7±0.5 degrees two-theta. See U.S. Pat. No. 9,957,282.

Polymorph E, as disclosed herein, is a polymorph of a free base of Compound I which exhibits an XRPD pattern comprising peaks at about 5.68 degrees two-theta with the margin of error of about ±0.50; about ±0.40; about ±0.30; about ±0.20; or about ±0.10; or less. In another embodiment, the XRPD of the crystalline Polymorph E further comprises peaks at about 12.2±0.5, 12.6±0.5, 25.4±0.5, and 27.6±0.5 degrees two-theta. See U.S. Pat. No. 9,957,282.

Additional characterization and methods of characterization the compound of the present invention are described below and in the Examples.

### Polymorph J

In one embodiment, Polymorph J of Compound I (free base) exhibits an XRPD comprising peaks at about 5.5 and about 11.0 degrees two-theta with the margin of error of about ±0.5; about ±0.4; about ±0.3; about ±0.2; about ±0.1; or less. In another embodiment, the XRPD of crystalline Polymorph J further comprises peaks at about 7.1 degrees two-theta with the margin of error of about ±0.5; about ±0.4; about ±0.3; about ±0.2; about ±0.1; or less. In further embodiment, the XRPD of crystalline Polymorph J further comprises peaks at about 17.7 and about ±26.7 degrees two-theta with the margin of error of about ±0.5; about ±0.4; about ±0.3; about ±0.2; about ±0.1; or less.

In one embodiment, Polymorph J of Compound I (free base) exhibits an XRPD comprising peaks at 5.5±0.5 and 11.0±0.5 degrees two-theta. In other embodiments, Compound I Polymorph J exhibits an XRPD pattern comprising peaks at 5.5±0.5, 7.1±0.5 and 11.0±0.5 degrees two-theta. In other embodiments, Compound I Polymorph J exhibits an XRPD pattern comprising peaks at 5.5±0.5, 7.1±0.5, 11.0±0.5, 17.7±0.5 and 26.7±0.5 degrees two-theta.

In one embodiment, Polymorph J of Compound I (free base) exhibits an XRPD comprising peaks at 5.5±0.5 and 11.0±0.5 degrees two-theta, which is the peak with the most intensity. In other embodiments, Compound I Polymorph J exhibits an XRPD pattern further comprises peaks at 7.1±0.5 and 11.0±0.5 degrees two-theta, wherein in each peak has at least 30% intensity of the peak at 5.5±0.5 degrees two-theta. In other embodiments, Compound I Polymorph J exhibits an XRPD pattern further comprises peaks at 17.7±0.5 and 26.7±0.5 degrees two-theta, wherein each peak has at least 10% intensity of the peak at 5.5±0.5 degrees two-theta.

In yet another embodiment, the crystalline Compound I Polymorph J exhibits an XRPD comprising peaks shown in Table 1 below:

TABLE 1
XRPD Table of Polymorph J of Compound I (free base)

| 2Theta | d-value | Intensity (Cps) | Intensity (%) |
|---|---|---|---|
| 5.504 | 16.042 | 89 | 100 |
| 6.36 | 13.885 | 1.92 | 2.2 |
| 7.064 | 12.504 | 36.6 | 41.1 |
| 7.709 | 11.459 | 2.9 | 3.3 |
| 9.586 | 9.219 | 7.89 | 8.9 |
| 11.003 | 8.034 | 40.2 | 45.2 |
| 11.601 | 7.622 | 1.05 | 1.2 |
| 12.314 | 7.182 | 1.37 | 1.5 |
| 12.779 | 6.921 | 2.15 | 2.4 |
| 14.184 | 6.239 | 5.51 | 6.2 |
| 15.872 | 5.579 | 6.22 | 7 |
| 16.566 | 5.347 | 1.47 | 1.7 |
| 17.651 | 5.021 | 16.3 | 18.3 |
| 18.352 | 4.83 | 4.1 | 4.6 |
| 18.634 | 4.758 | 4.7 | 5.3 |
| 19.12 | 4.638 | 8.52 | 9.6 |
| 20.038 | 4.428 | 2.27 | 2.5 |
| 20.486 | 4.332 | 1.98 | 2.2 |
| 21.322 | 4.164 | 4.67 | 5.2 |
| 22.21 | 3.999 | 2.81 | 3.2 |
| 22.714 | 3.912 | 4.35 | 4.9 |
| 23.104 | 3.847 | 4.78 | 5.4 |
| 23.913 | 3.718 | 0.85 | 1 |
| 24.78 | 3.59 | 7.33 | 8.2 |
| 25.76 | 3.456 | 1.09 | 1.2 |
| 26.188 | 3.4 | 1.61 | 1.8 |
| 26.745 | 3.331 | 18.8 | 21.2 |
| 27.346 | 3.259 | 0.97 | 1.1 |
| 27.804 | 3.206 | 1.51 | 1.7 |
| 28.568 | 3.122 | 0.85 | 1 |
| 30.434 | 2.935 | 1.05 | 1.2 |
| 30.815 | 2.899 | 1.32 | 1.5 |
| 32.099 | 2.786 | 1.6 | 1.8 |
| 33.31 | 2.688 | 1.33 | 1.5 |
| 34.726 | 2.581 | 0.58 | 0.7 |
| 35.731 | 2.511 | 1.81 | 2 |
| 36.032 | 2.491 | 0.92 | 1 |
| 36.644 | 2.45 | 1.18 | 1.3 |
| 37.146 | 2.418 | 0.68 | 0.8 |
| 37.588 | 2.391 | 0.88 | 1 |
| 38.135 | 2.358 | 0.6 | 0.7 |
| 39.296 | 2.291 | 0.9 | 1 |
| 39.841 | 2.261 | 1.33 | 1.5 |

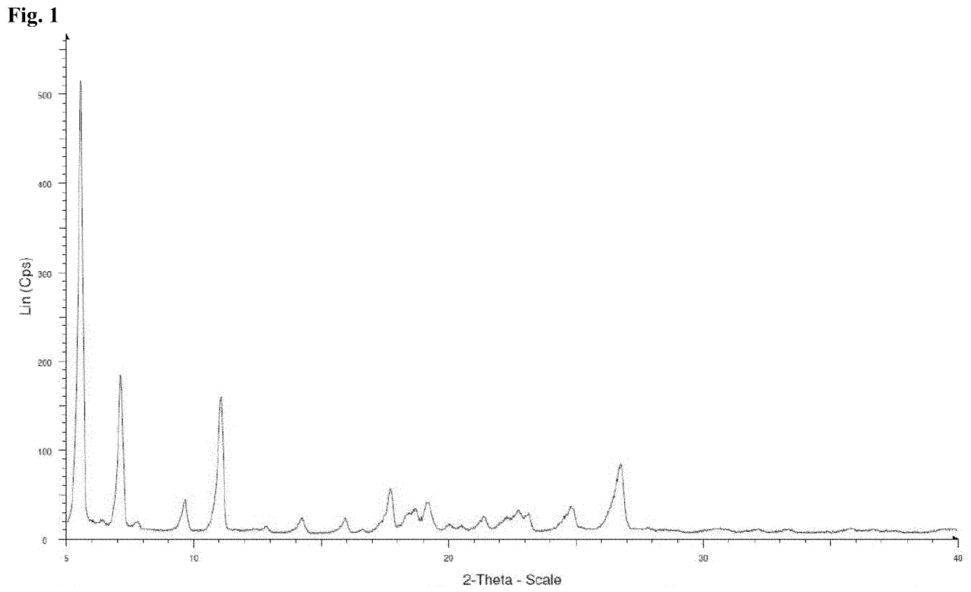

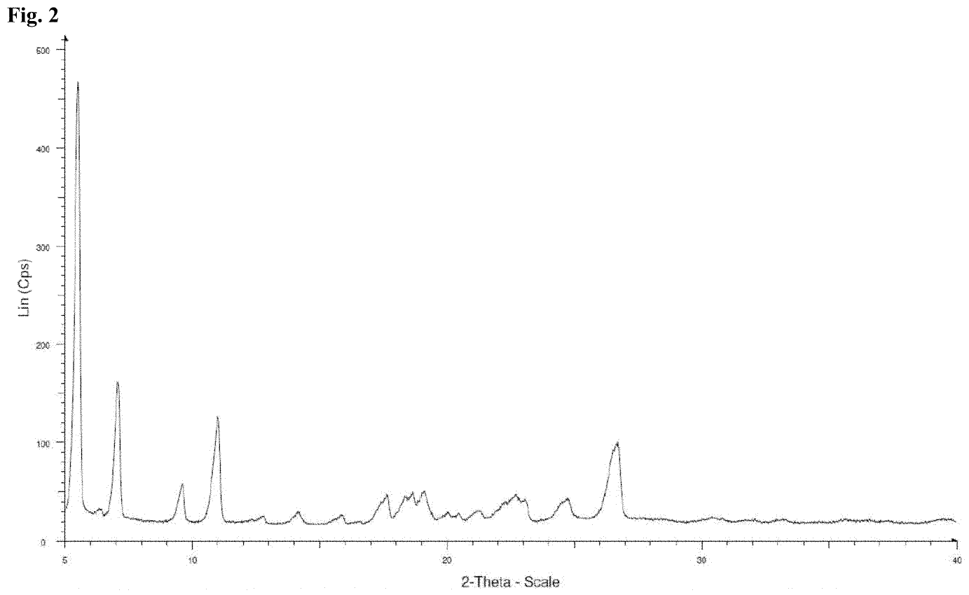

In one specific embodiment, the crystalline Polymorph J of Compound I (free base) exhibits an XRPD pattern that is substantially similar to FIG. 1. In one specific embodiment, the crystalline Polymorph J of Compound I (free base) exhibits an XRPD pattern that is substantially similar to FIG. 2. FIGS. 1 and 2 represents XRPD pattern of Polymorph J obtained from different synthesis batches.

In one embodiment, the crystalline Compound I (free base) Polymorph J exhibits a DSC thermogram comprising an endotherm peak maximum in between about 200.0° C. to about 202.0° C. with the error of margin of about ±2.5; about ±2.0; about ±1.5; about ±1.0; about ±0.5° C.; or less. In another embodiment, Polymorph J exhibits a DSC thermogram comprising an endotherm peak maximum in between 200.0° C.±0.5° C. to about 202.0±0.5° C.

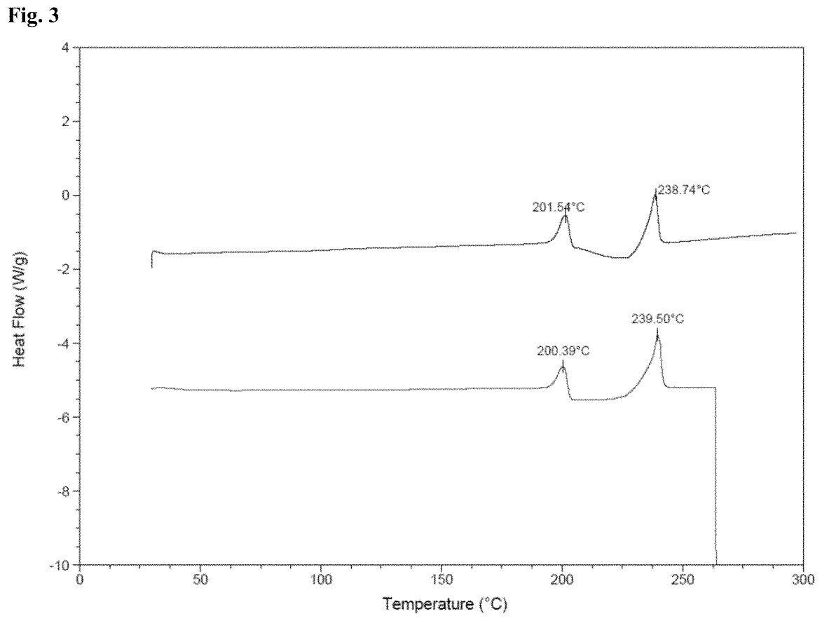

In one embodiment, Polymorph J exhibits a DSC thermogram comprising an endotherm peak maximum in between about ±238.0° C. to about ±246.0° C. with the error of margin of about ±2.5; about ±2.0; about ±1.5; about ±1.0; about ±0.5° C.; or less. In another embodiment, Polymorph J exhibits a DSC thermogram comprising an endotherm peak maximum in between 238.0° C.±0.5° C. to about 246.0±0.5° C. In another embodiment, Polymorph J exhibits a DSC thermogram comprising an endotherm peak maximum in between 238.0° C.±0.5° C. to about 245.0±0.5° C. In another embodiment, Polymorph J exhibits a DSC thermogram comprising an endotherm peak maximum in between 238.0° C.±0.5° C. to about 240.0±0.5° C. In another embodiment, Polymorph J exhibits a DSC thermogram comprising an endotherm peak maximum in between 238.0° C.±0.5° C. to about 239.0±0.5° C. In one embodiment, Polymorph J exhibits a DSC thermogram that is substantially similar to FIG. 3.

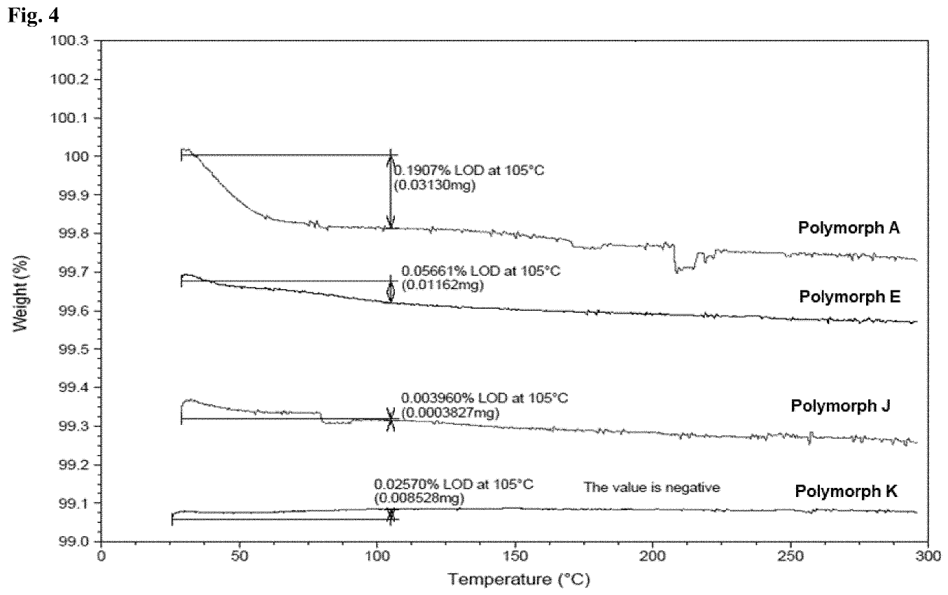

In one embodiment, Polymorph J exhibits a thermogravimetric analysis (TGA) thermogram substantially similar to FIG. 4 (third line from the top). In one embodiment, Polymorph J exhibits about ±0.004% weight loss (loss on drying, LOD) at about ±105° C. when TGA analysis is performed from ambient temperature to 300° C.

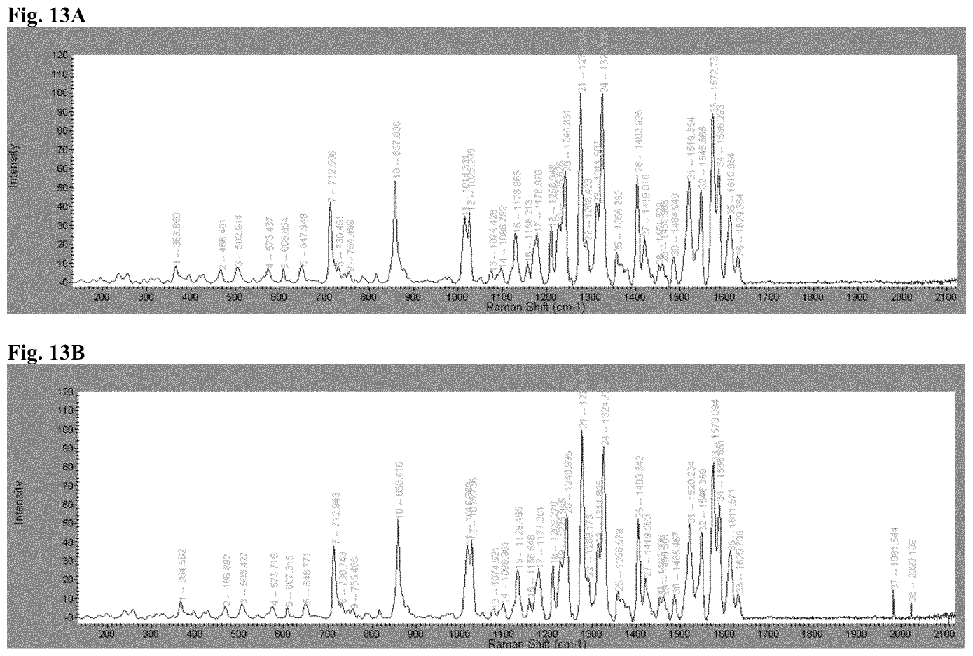

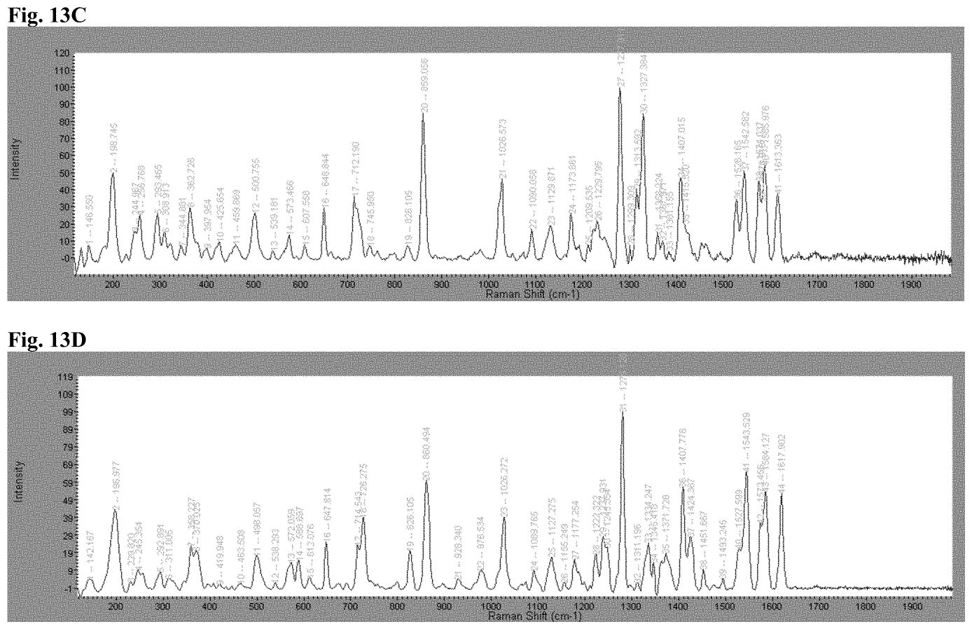

In one embodiment, Polymorph J of Compound I (free base) exhibits a Raman spectrum comprising a peak at about 1014±5 cm⁻¹. In one embodiment, Polymorph J exhibits a Raman spectrum that is substantially similar to FIG. 13A.

### Polymorph K

In one embodiment, Polymorph K of Compound I (free base) exhibits an XRPD comprising peaks at about 5.2 and about 25.5 degrees two-theta with the margin of error of about ±0.5; about ±0.4; about ±0.3; about ±0.2; about ±0.1; or less. In another embodiment, the XRPD of crystalline Polymorph K further comprises peaks at about 11.4 degrees two-theta with the margin of error of about ±0.5; about ±0.4; about ±0.3; about ±0.2; about ±0.1; or less. In further embodiment, the XRPD of crystalline Polymorph K further comprises peaks at about 14.7 and about 23.4 degrees two-theta with the margin of error of about ±0.5; about ±0.4; about ±0.3; about ±0.2; about ±0.1; or less.

In one embodiment, Polymorph K of Compound I (free base) exhibits an XRPD comprising peaks at 5.2±0.5 and 25.5±0.5 degrees two-theta. In another embodiment, the XRPD of crystalline Polymorph K comprises peaks at 5.2±0.5, 11.4±0.5 and 25.5±0.5 degrees two-theta. In further embodiment, the XRPD of crystalline Polymorph K comprises peaks at 5.2±0.5, 11.4±0.5, 14.7±0.5, 23.4±0.5 and 25.5±0.5 degrees two-theta.

In one embodiment, Polymorph K of Compound I (free base) exhibits an XRPD comprising peaks at 5.2±0.5 degrees two-theta, which is the peak with the most intensity. In another embodiment, the XRPD of crystalline Polymorph K further comprises peaks at 25.5±0.5 degrees two-theta, wherein the peak has at least 4% intensity of the peak at 5.2±0.5 degrees two-theta. In further embodiment, the XRPD of crystalline Polymorph K further comprises peaks at 11.4±0.5, 14.7±0.5, and 23.4±0.5 degrees two-theta, wherein the peak has at least 3% intensity of the peak at 5.2±0.5 degrees two-theta.

In yet another embodiment, the crystalline Compound I Polymorph K exhibits an XRPD comprising peaks shown in Table 1 below:

TABLE 2
XRPD Table of Polymorph K of Compound I (free base)

| 2Theta | d-value | Intensity (Cps) | Intensity (%) |
|---|---|---|---|
| 5.185 | 17.029 | 258 | 100 |
| 5.74 | 15.385 | 9.78 | 3.8 |
| 7.375 | 11.977 | 3.92 | 1.5 |
| 8.57 | 10.309 | 6.05 | 2.3 |
| 9.596 | 9.21 | 5.84 | 2.3 |
| 10.343 | 8.546 | 3.3 | 1.3 |
| 10.906 | 8.106 | 1.56 | 0.6 |
| 11.401 | 7.755 | 11.3 | 4.4 |
| 11.675 | 7.574 | 4.53 | 1.8 |
| 12.569 | 7.037 | 3.11 | 1.2 |
| 13.299 | 6.652 | 0.87 | 0.3 |
| 13.771 | 6.425 | 2.52 | 1 |
| 14.163 | 6.248 | 3.11 | 1.2 |
| 14.654 | 6.04 | 10.5 | 4.1 |
| 15.486 | 5.717 | 6.23 | 2.4 |
| 16.217 | 5.461 | 5.06 | 2 |
| 17.435 | 5.082 | 2.05 | 0.8 |
| 17.846 | 4.966 | 2.2 | 0.9 |
| 18.158 | 4.882 | 2.12 | 0.8 |
| 18.456 | 4.803 | 2.54 | 1 |
| 19.236 | 4.61 | 7.86 | 3 |
| 19.9 | 4.458 | 1.06 | 0.4 |
| 20.315 | 4.368 | 2.45 | 0.9 |
| 20.557 | 4.317 | 2.94 | 1.1 |
| 21.371 | 4.154 | 3.19 | 1.2 |
| 21.827 | 4.069 | 1.34 | 0.5 |
| 22.185 | 4.004 | 2.94 | 1.1 |
| 22.85 | 3.889 | 3.71 | 1.4 |
| 23.373 | 3.803 | 11 | 4.2 |
| 23.87 | 3.725 | 5.05 | 2 |
| 24.636 | 3.611 | 3.61 | 1.4 |
| 24.859 | 3.579 | 2.51 | 1 |
| 25.511 | 3.489 | 15.2 | 5.9 |
| 25.968 | 3.428 | 3.88 | 1.5 |
| 26.579 | 3.351 | 2.2 | 0.9 |
| 27.622 | 3.227 | 1.72 | 0.7 |
| 28.47 | 3.133 | 5.47 | 2.1 |
| 28.849 | 3.092 | 4.15 | 1.6 |
| 29.43 | 3.033 | 1.72 | 0.7 |
| 29.659 | 3.01 | 1.38 | 0.5 |
| 30.066 | 2.97 | 0.91 | 0.4 |
| 30.73 | 2.907 | 1.69 | 0.7 |
| 31.074 | 2.876 | 1.17 | 0.5 |
| 31.791 | 2.813 | 1.42 | 0.5 |
| 32.503 | 2.753 | 0.9 | 0.3 |
| 33.065 | 2.707 | 1.49 | 0.6 |
| 34.93 | 2.567 | 1.13 | 0.4 |
| 35.692 | 2.514 | 0.97 | 0.4 |
| 37.128 | 2.42 | 1 | 0.4 |
| 38.917 | 2.312 | 1.05 | 0.4 |
| 39.818 | 2.262 | 1.94 | 0.7 |

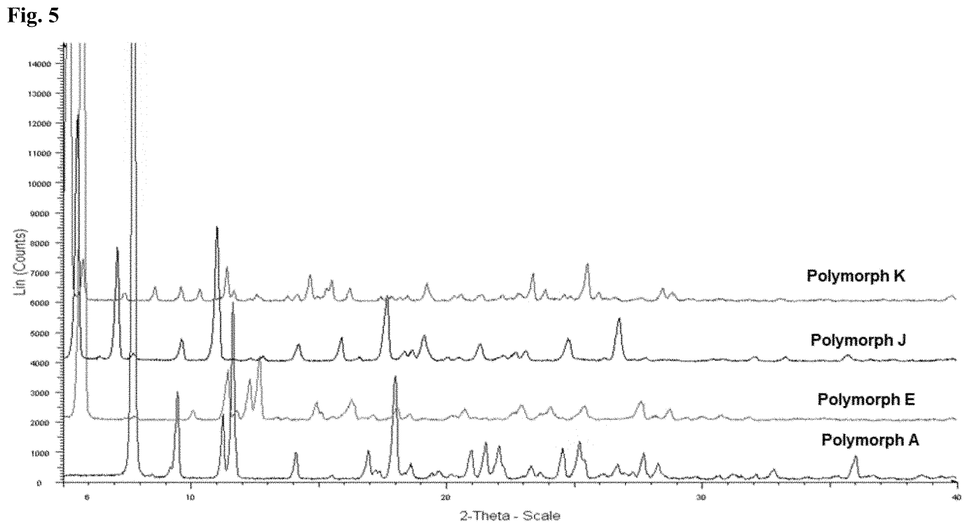

In one specific embodiment, the crystalline Polymorph K of Compound I (free base) exhibits an XRPD pattern that is substantially similar to FIG. 5 (top line).

In one embodiment, the crystalline Compound I (free base) Polymorph K exhibits a DSC thermogram comprising an endotherm peak maximum in between about 144.0° C. to about 150.0° C. with the error of margin of about ±2.5; about ±2.0; about ±1.5; about ±1.0; about ±0.5° C.; or less. In another embodiment, Polymorph K exhibits a DSC thermogram comprising an endotherm peak maximum in between 144.0° C.±0.5° C. to about 150.0±0.5° C. 10125 In one embodiment, Polymorph K exhibits a DSC thermogram comprising an endotherm peak maximum in between about 231.0° C. to about 238.0° C. with the error of margin of about ±2.5; about ±2.0; about ±1.5; about ±1.0; about ±0.5° C.; or less. In another embodiment, Polymorph K exhibits a DSC thermogram comprising an endotherm peak maximum in between 231.0° C. 0.5° C. to about 238.0±0.5° C.

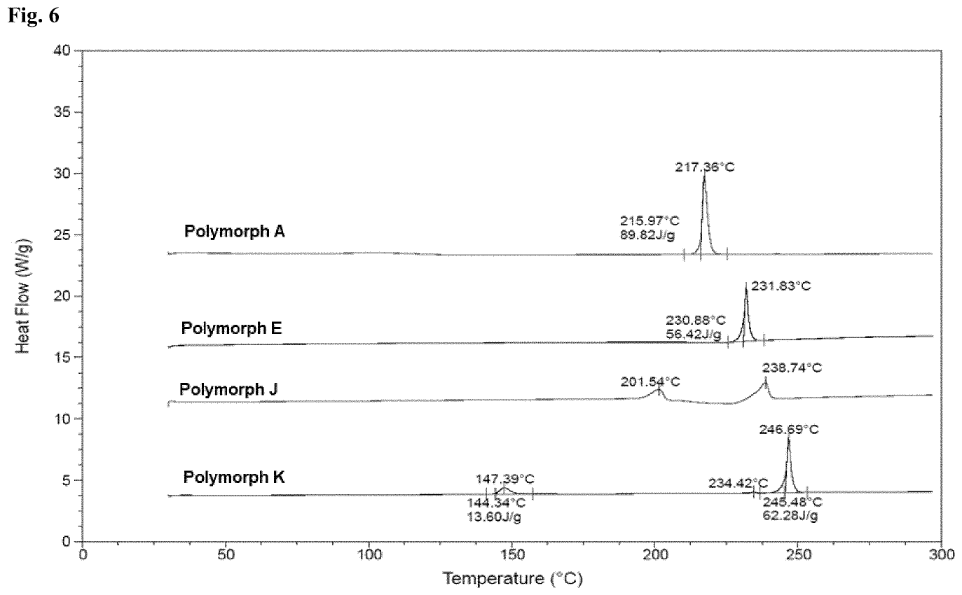

In one embodiment, Polymorph K exhibits a DSC thermogram comprising an endotherm peak maximum in between about 242.0° C. to about 250.0° C. with the error of margin of about ±2.5; about ±2.0; about ±1.5; about ±1.0; about ±0.5° C.; or less. In another embodiment, Polymorph K exhibits a DSC thermogram comprising an endotherm peak maximum in between 242.0° C. 0.5° C. to about 250.0±0.5° C. In one embodiment, Polymorph J exhibits a DSC thermogram that is substantially similar to FIG. 6 (bottom line).

In one embodiment, Polymorph K exhibits a thermogravimetric analysis (TGA) thermogram substantially similar to FIG. 4 (fourth line from the top).

In one embodiment, Polymorph K of Compound I (free base) exhibits a Raman spectrum comprising a peak at about 1015±5 cm¹. In one embodiment, Polymorph K exhibits a Raman spectrum that is substantially similar to FIG. 13B.

Pharmaceutical Formulations

In another embodiment, the present invention provides a pharmaceutical composition comprising a therapeutically effective amount of a crystalline form of Compound I, or a pharmaceutically acceptable salt, ester, and/or solvate thereof, as disclosed herein, as the active ingredient, combined with a pharmaceutically acceptable excipient or carrier. The excipients are added to the formulation for a variety of purposes.

In one embodiment, the present disclosure relates to solid formulation where the crystalline form of Compound I is maintained. In some embodiments, the present disclosure relates to formulation of various types as disclosed herein, prepared from a crystalline form of Compound I.

Diluents may be added to the formulations of the present invention. Diluents increase the bulk of a solid pharmaceutical composition, and may make a pharmaceutical dosage form containing the composition easier for the patient and care giver to handle. Diluents for solid compositions include, for example, microcrystalline cellulose (e.g., AVICEL), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT (r)), potassium chloride, powdered cellulose, sodium chloride, sorbitol, and talc. Diluents for liquid compositions include, but are not limited to, water, aqueous solutions of saccharides and/or sugar alcohols (e.g., glucose solution, dextrose solution, lactose solution, maltose solution, fructose solution), saline solution, and other aqueous medium.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g., carbopol), carboxymethylcellulose sodium, dextrin, ethyl cellulose, gelatin, guar gum, gum tragacanth, hydrogenated vegetable oil, hydroxyethyl cellulose, hydroxypropyl cellulose (e.g., KLUCEL), hydroxypropyl methyl cellulose (e.g., METHOCEL), liquid glucose, magnesium aluminum silicate, maltodextrin, methylcellulose, polymethacrylates, povidone (e.g., KOLLIDON, PLASDONE), pregelatinized starch, sodium alginate, and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g., AC-DI-SOL and PRIMELLOSE), colloidal silicon dioxide, croscarmellose sodium, crospovidone (e.g., KOLLIDON and POLYPLASDONE), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB), potato starch, and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

In some embodiments, the crystalline form of Compound I is maintained through the tableting process, including being under pressure from a punch and dye.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In liquid pharmaceutical compositions may be prepared using the crystalline forms of the present invention and any other solid excipients where the components are dissolved or suspended in a liquid carrier such as water, vegetable oil, alcohol, polyethylene glycol, propylene glycol, or glycerin.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as aspartame, lactose, sorbitol, saccharin, sodium saccharin, sucrose, aspartame, fructose, mannitol, and invert sugar may be added to improve the taste.

Preservatives and chelating agents such as alcohol, sodium benzoate, butylated hydroxyl toluene, butylated hydroxyanisole, and ethylenediamine tetraacetic acid may be added at levels safe for ingestion to improve storage stability.

A liquid composition may also contain a buffer such as gluconic acid, lactic acid, citric acid or acetic acid, sodium gluconate, sodium lactate, sodium citrate, or sodium acetate. Selection of excipients and the amounts used may be readily determined by the formulation scientist based upon experience and consideration of standard procedures and reference works in the field.

A liquid composition can be for injection. A liquid composition may contain sterile diluent, such as but not limited to, water, glucose solution, dextrose solution, sucrose solution, or saline solution.

In a liquid composition of the present disclosure, the pH of the composition can be adjusted using acidifying agent and/or alkalizing agent. In some embodiments, the pH of the composition can be adjusted with aqueous HCl and/or aqueous NaOH. In some embodiments, the pH of the composition is in the range from about 4.0 to about 6.0, including all values and subranges therebetween.

In some embodiments, the liquid composition is prepared under anaerobic conditions. In some embodiments, the materials used to prepare the liquid composition are sparged with nitrogen before use. In some embodiments, the liquid composition is sparged with nitrogen until soluble oxygen level reaches less than 1.0 ppm. In some embodiments, the liquid composition is prepared and sealed or capped under nitrogen.

The solid compositions of the present invention include powders, granules, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and lozenges, as well as liquid syrups, suspensions, aerosols and elixirs.

The dosage form of the present invention may be a capsule containing the composition, preferably a powdered or granule solid composition of the invention, within either a hard or soft shell. The shell may be made from gelatin and optionally contain a plasticizer such as glycerin and sorbitol, and an opacifying agent or colorant.

A composition for tableting or capsule filling may be prepared by wet granulation. In wet granulation, some or all of the active ingredients and excipients in powder form are blended and then further mixed in the presence of a liquid, typically water that causes the powders to clump into granules. The granules are screened and/or milled, dried and then screened and/or milled to the desired particle size. The granules may be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granules that were described with reference to tableting; however, they are not subjected to a final tableting step.

In one embodiment, the crystalline form of Compound I, or a pharmaceutically acceptable salt and/or solvate thereof, is reconstituted prior to administration in pharmaceutically acceptable carrier or solvent. In one embodiment, the reconstituted solution formulation comprising Compound I, or a pharmaceutically acceptable salt and/or solvate thereof, is administered by an IV.

The active ingredient and excipients may be formulated into compositions and dosage forms according to methods known in the art.

In one embodiment, a dosage form may be provided as a kit comprising crystalline form of Compound I and pharmaceutically acceptable excipients and carriers as separate components. In some embodiments, the dosage form kit allow physicians and patients to formulate an oral solution or injection solution prior to use by dissolving, suspending, or mixing the crystalline form of Compound I with pharmaceutically acceptable excipients and carriers. In one embodiment, a dosage form kit which provides crystalline form of Compound I has improved stability of Compound I compared to pre-formulated liquid formulations of Compound I.

A dosage form of the present invention may contain at least one of crystalline form of Compound I or a pharmaceutically acceptable salt or ester thereof, as disclosed herein, in an amount of about 5 mg to about 500 mg, or any value in between. That is, a dosage form of the present invention may contain a crystalline form of Compound I or a pharmaceutically acceptable salt or ester thereof (such as Polymorph J or K), in an amount of about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 125 mg, 130 mg, 140 mg, 150 mg, 160 mg, 170 mg, 175 mg, 180 mg, 190 mg, 200 mg, 210 mg, 220 mg, 225 mg, 230 mg, 240 mg, 250 mg, 260 mg, 270 mg, 275 mg, 280 mg, 290 mg, 300 mg, 310 mg, 320 mg, 325 mg, 330 mg, 340 mg, 350 mg, 360 mg, 370 mg, 375 mg, 380 mg, 390 mg, 400 mg, 410 mg, 420 mg, 425 mg, 430 mg, 440 mg, 450 mg, 460 mg, 470 mg, 475 mg, 480 mg, 490 mg, or 500 mg.

A dosage form of the present invention may contain at least one of crystalline form of Compound I or a pharmaceutically acceptable salt or ester thereof, as disclosed herein, such that the total amount of Compound I (can be in various forms) totals about 5 mg to about 500 mg, or any value in between. That is, a dosage form of the present invention comprise a crystalline form of Compound I or a pharmaceutically acceptable salt or ester thereof and optionally other forms of Compound I such that the total amount of Compound I is in an amount of about: 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 125 mg, 130 mg, 140 mg, 150 mg, 160 mg, 170 mg, 175 mg, 180 mg, 190 mg, 200 mg, 210 mg, 220 mg, 225 mg, 230 mg, 240 mg, 250 mg, 260 mg, 270 mg, 275 mg, 280 mg, 290 mg, 300 mg, 310 mg, 320 mg, 325 mg, 330 mg, 340 mg, 350 mg, 360 mg, 370 mg, 375 mg, 380 mg, 390 mg, 400 mg, 410 mg, 420 mg, 425 mg, 430 mg, 440 mg, 450 mg, 460 mg, 470 mg, 475 mg, 480 mg, 490 mg, or 500 mg.

It is not necessary that the formulations of the present invention contain only one crystalline form of Compound I. The crystalline forms of the present invention may be used in pharmaceutical formulations or compositions as single components or mixtures together with other crystalline forms of Compound I (such as Polymorphs J and A). In one embodiment, pharmaceutical formulations or compositions of the present invention contain 25-100% or 50-100% by weight, of at least one of crystalline form of Compound I as described herein, in the formulation or composition.

In one embodiment, the preparation of any one of the compositions, formulations, dosage forms as disclosed herein can be prepared under anaerobic conditions.

In one embodiment, such dosage amount is administered to a patient as a daily dose either in a single dose or in divided portions served multiple times a day, such as twice, three times, or four times a day.

Therapeutic Use

The present invention also provides treatment of disorders related to proliferation of cells. In one aspect, there is provided a method for selectively activating p53 protein comprising contacting a cell afflicted by disorder related to cell proliferation with the present compound. In one embodiment, the method comprises contacting cancer and/or tumor cells with the crystalline form of Compound I, or a pharmaceutically acceptable salt, ester, and/or solvate thereof, as disclosed herein. In another embodiment, the method of contacting cancer and/or tumor cells with the crystalline form of Compound I, or a pharmaceutically acceptable salt, ester, and/or solvate thereof, as disclosed herein, may induce cell apoptosis or alleviate or prevent the progression of the disorder.

In one embodiment, the present invention provides a method for stabilizing G-quadruplex (G4) comprising contacting a cell afflicted by disorder related to cell proliferation with at least one compound of the invention. In one embodiment, the method comprises contacting cancer and/or tumor cells with at least one compound of the invention. In another embodiment, the method of contacting cancer and/or tumor cells with at least one compound of the present invention, may induce cell apoptosis or alleviate or delay the progression of the disorder.

In one embodiment, the compound of the present invention, can be administered in an amount effective to stabilize G4 in cancer and/or tumor cells, which may lead to cell death or apoptosis.

The present invention also provides methods of treating, preventing, ameliorating and/or alleviating the progression of disorders or conditions characterized by cell proliferation in a subject. More particularly, the methods of the present invention involve administration of an effective amount of the crystalline form of the quinolone compounds described herein, in a subject to treat a disorder or a condition characterized by cell proliferation. The crystalline form can be administered in an amount effective selectively activate p53 proteins in cancer and/or tumor cells, which may lead to cell death or apoptosis. The terms “subject” and “patient” are used interchangeably throughout the present application.

In one embodiment, the present invention relates to method of treating cancer comprising administering to a subject in need thereof an effective amount of the compound of the present invention. In one embodiment, cancer treated or ameliorated by the method as disclosed herein may be selected from Acute Lymphoblastic Leukemia, Acute Myeloid Leukemia, Adrenocortical Carcinoma, AIDS-Related Cancers, Kaposi Sarcoma, Lymphoma, Anal Cancer, Appendix Cancer, Astrocytomas, Childhood Atypical Teratoid/Rhabdoid Tumor, Basal Cell Carcinoma, Skin Cancer (Nonmelanoma), Childhood Bile Duct Cancer, Extrahepatic Bladder Cancer, Bone Cancer, Ewing Sarcoma Family of Tumors, Osteosarcoma and Malignant Fibrous Histiocytoma, Brain Stem Glioma, Brain Tumors, Embryonal Tumors, Germ Cell Tumors, Craniopharyngioma, Ependymoma, Bronchial Tumors, Burkitt Lymphoma (Non-Hodgkin Lymphoma), Carcinoid Tumor, Gastrointestinal Carcinoma of Unknown Primary, Cardiac (Heart) Tumors, Lymphoma, Primary, Cervical Cancer, Childhood Cancers, Chordoma, Chronic Lymphocytic Leukemia, Chronic Myelogenous Leukemia, Chronic Myeloproliferative Neoplasms Colon Cancer, Colorectal Cancer, Cutaneous T-Cell Lymphoma, Ductal Carcinoma In Situ, Endometrial Cancer, Ependymoma, Esophageal Cancer, Esthesioneuroblastoma, Ewing Sarcoma, Extracranial Germ Cell Tumor, Extragonadal Germ Cell Tumor, Extrahepatic Bile Duct Cancer, Eye Cancer, Intraocular Melanoma, Retinoblastoma, Fibrous Histiocytoma of Bone, Malignant, and Osteosarcoma, Gallbladder Cancer, Gastric (Stomach) Cancer, Gastrointestinal Carcinoid Tumor, Gastrointestinal Stromal Tumors, Extragonadal Cancer, Ovarian Cancer, Testicular Cancer, Gestational Trophoblastic Disease, Glioma, Brain Stem Cancer, Hairy Cell Leukemia, Head and Neck Cancer, Heart Cancer, Hepatocellular (Liver) Cancer, Histiocytosis, Langerhans Cell Cancer, Hodgkin Lymphoma, Hypopharyngeal Cancer, Intraocular Melanoma, Islet Cell Tumors, Pancreatic Neuroendocrine Tumors, Kaposi Sarcoma, Kidney Cancer, Renal Cell Cancer, Wilms Tumor and Other Childhood Kidney Tumors, Langerhans Cell Histiocytosis, Laryngeal Cancer, Leukemia, Chronic Lymphocytic Cancer, Chronic Myelogenous Cancer, Hairy Cell Cancer, Lip and Oral Cavity Cancer, Liver Cancer (Primary), Lobular Carcinoma In Situ (LCIS), Lung Cancer, Non-Small Cell Cancer, Small Cell Cancer, Lymphoma, Cutaneous T-Cell (Mycosis Fungoides and Sézary Syndrome), Hodgkin Cancer, Non-Hodgkin Cancer, Macroglobulinemia, Waldenström, Male Breast Cancer, Malignant Fibrous Histiocytoma of Bone and Osteosarcoma, Melanoma, Intraocular (Eye) Cancer, Merkel Cell Carcinoma, Mesothelioma, Malignant, Metastatic Squamous Neck Cancer with Occult Primary, Midline Tract Carcinoma Involving NUT Gene, Mouth Cancer, Multiple Endocrine Neoplasia Syndromes, Multiple Myeloma/Plasma Cell Neoplasm, Mycosis Fungoides, Myelodysplastic Syndromes, Myelodysplastic/Myeloproliferative Neoplasms, Myelogenous Leukemia, Chronic, Myeloid Leukemia, Acute, Myeloma Multiple, Chronic Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Oral Cancer, Oral Cavity Cancer, Lip and Oropharyngeal Cancer, Osteosarcoma and Malignant Fibrous Histiocytoma of Bone, Epithelial Cancer, Low Malignant Potential Tumor, Pancreatic Cancer, Pancreatic Neuroendocrine Tumors (Islet Cell Tumors), Papillomatosis, Paraganglioma, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Primary Central Nervous System Lymphoma, Rectal Cancer, Renal Cell (Kidney) Cancer, Retinoblastoma, Rhabdomyosarcoma, Salivary Gland Cancer, Sarcoma, Ewing Cancer, Kaposi Cancer, Osteosarcoma (Bone Cancer), Soft Tissue Cancer, Uterine Cancer, Sézary Syndrome, Skin Cancer, Childhood Melanoma, Merkel Cell Carcinoma, Nonmelanoma, Small Cell Lung Cancer, Small Intestine Cancer, Soft Tissue Sarcoma, Squamous Cell Carcinoma, Skin Cancer (Nonmelanoma), Childhood Squamous Neck Cancer with Occult Primary, Metastatic Cancer, Stomach (Gastric) Cancer, T-Cell Lymphoma, Cutaneous Cancer, Testicular Cancer, Throat Cancer, Thymoma and Thymic Carcinoma, Thyroid Cancer, Transitional Cell Cancer of the Renal Pelvis and Ureter, Unknown Primary, Carcinoma of Childhood, Unusual Cancers of Childhood, Urethral Cancer, Uterine Cancer, Endometrial Cancer, Uterine Sarcoma, Vaginal Cancer, Vulvar Cancer, Waldenström Macroglobulinemia, Wilms Tumor, or Women's Cancers.

Additionally, disclosed are methods for treating cancers, cancer cells, tumors, or tumor cells. Non limiting examples of cancer that may be treated by the methods of this disclosure include cancer or cancer cells of: large intestine, breast, lung, liver, pancreas, lymph node, colon, rectum, prostate, brain, head and neck, skin, ovary, cervical, thyroid, bladder, kidney, and blood and heart (e.g., leukemia, lymphoma, and carcinoma). Non limiting examples of tumors that may be treated by the methods of this disclosure include tumors and tumor cells of: large intestine, breast, lung, liver, pancreas, lymph node, colon, rectum, prostate, brain, head and neck, skin, kidney, and blood and heart (e.g., leukemia, lymphoma, and carcinoma), uterine, gastrointestine, larynx, and oral cavity.

In one embodiment, cancer treated or ameliorated by any one of the methods as disclosed herein may be selected from the group consisting of: heme cancer (hematologic malignancies), colorectal cancer, breast cancer, lung cancer, liver cancer, ovarian cancer, cervical cancer, Ewing's sarcoma, pancreatic cancer, cancer of the lymph nodes, colon cancer, prostate cancer, brain cancer, cancer of the head and neck, skin cancer, kidney cancer, cancer of the heart, uterine cancer, gastrointestinal malignancies, and carcinomas of the larynx and oral cavity. In some embodiments, the cancer treated or ameliorated by the method is selected from the group consisting of uterine cancer, gastrointestinal malignancies, and carcinomas of the larynx and oral cavity. In one embodiment, cancer treated or ameliorated by the method is hematologic malignancies which is selected from the group consisting of: leukemia, lymphoma, myeloma, and multiple myeloma. In one embodiment, cancer treated or ameliorated by any one of the methods as disclosed herein may be selected from the group consisting of: hematologic malignancies, colorectal cancer, breast cancer, lung cancer, liver cancer, ovarian cancer, cervical cancer, Ewing's sarcoma, pancreatic cancer, cancer of the lymph nodes, colon cancer, prostate cancer, brain cancer, cancer of the head and neck, skin cancer, kidney cancer, osteosarcoma, and cancer of the heart. In one embodiment, cancer treated or ameliorated by the method is heme cancer which is selected from the group consisting of leukemia, lymphoma, myeloma, and multiple myeloma.

In one embodiment, the compound of the invention is useful for treating breast cancer. In one embodiment, the compound of the invention is useful for treating ovarian cancer. In one embodiment, the compound of the invention is useful for treating solid tumors. In one embodiment, the compound of the invention is useful for treating pancreatic cancer. In one embodiment, the compound of the invention is useful for treating pancreatic tumor. In one embodiment, the compound of the invention is useful for treating non-small cell lung cancer. In one embodiment, the compound of the invention is useful for treating hematologic malignancies. In one embodiment, the compound of the invention is useful for treating hematologic malignancies.

In one embodiment, cancer treated or ameliorated by any one of the methods as disclosed herein can be wherein the subject has a mutation in a DNA repair gene. In a specific embodiment, the DNA repair gene is a homologous recombinant gene. In another embodiment, the DNA repair gene is a gene in the homologous recombination (HR) dependent deoxynbounucleic acid (DNA) double strand break (DSB) repair pathway. In a specific embodiment, the DNA repair gene is a homologous recombinant (HR) or non-homologous end joining (NHEJ) gene. In another embodiment, the DNA repair gene is a gene in the homologous recombination (HR) or non-homologous end joining (NHEJ) dependent deoxyribonucleic acid (DNA) double strand break (DSB) repair pathway. In another method, the DNA repair gene is one or more genes selected from the group consisting of BRCA-1, BRCA-2, ATM, ATR, CHK1, CHK2, Rad51, RPA and XRCC3.

In one embodiment of any one of the methods as disclosed herein, the subject has a mutation in one or more genes in the HR pathway, Fanconi anemia pathway, mismatch repair pathway, ATM pathway, cell cycle pathway, p53 signaling pathway, polymerase pathway, topoisomerase pathway. In one embodiment, the subject has a mutation in one or more genes having a function in HR repair, ATM pathway, cell cycle, topoisomerase, double-strand break repair, excision repair, C-Myb transcription factor network, p-53 signaling, and/or apoptosis or genomic stability. In one embodiment, the subject has a mutation in one or more genes selected from BRCA1, BRCA2, PTEN, ATM, CHEK1, TOP2A, ABL1, PER1, RAD51, ERCC5, NBN, TRIM28, SETMAR, RAD54L, EYA1, and TP53. In one embodiment, the subject has a mutation in one or more genes selected from ARID1A, ATM, ATR, BAP1, BARD1, BLM, BRCA, BRCA2, CHEK1, CHEK2, ERCC3, FANCG, FANCL, FANCL, HELQ, MLHI, MRE1A, MSH2, MSH6, MUTYH, PMS1, POLE, POLRIB, PTEN, RAD17, RAD51D, RAD54L, TOP3A, and/or WRN.

In one embodiment, the subject has a mutation in one or more genes selected from BRCA1, BRCA2, TP53, and PALB2. In another embodiment, the subject has a mutation in BRCA1, and/or BRCA2 genes, and/or other genes of the HR pathway. In some embodiments, the mutation is a somatic mutation. In other embodiments, the mutation is a germline mutation.

In one embodiment, Compound I or a pharmaceutically acceptable salt thereof's efficacy is associated with a mutation or a copy number loss of a gene in the HR pathway or the Fanconi anemia pathway, wherein the gene is selected from: ARID1A, ATM, ATR, BAP1, BARD1, BLM, BRCA1, BRCA2, FANCG, FANCL, FANCL, HELQ, MRE1A, NBN, PALB2, PTEN, RAD51, RAD51D, RAD54L, and/or WRN. In one embodiment, Compound I or a pharmaceutically acceptable salt thereof's efficacy is associated with a mutation or a copy number loss of HR pathway gene BRCA2 and/or PALB2.

In another embodiment, cancer treated or ameliorated by the method comprises cancer cells harboring defects in BRCA1 gene (breast cancer type 1), BRCA2 (breast cancer type 2), and/or other members of the homologous recombination pathway. In another embodiment, the cancer cells are deficient in BRCA1 and/or BRCA2. In another embodiment, the cancer cells are homozygous for a mutation in BRCA1 and/or BRCA2. In another embodiment, the cancer cells are heterozygous for a mutation in BRCA1 and/or BRCA2. In some embodiments, the cancer cells are deficient in germline BRCA1 and/or BRCA2. In another embodiment, the cancer cells are deficient in somatic BRCA1 and/or BRCA2.

In one embodiment, cancer treated or ameliorated by any one of the methods as disclosed herein is BRCA2 deficient. In another embodiment, Compound I or a pharmaceutically acceptable salt or solvate thereof or the compound of the present invention induces more apoptotic cell death in BRCA2 deficient or BRCA2 knockout cells relative to BRCA2 proficient or BRCA2 wild type cells. In one embodiment, Compound I or a pharmaceutically acceptable salt or solvate thereof or the compound of the present invention is selectively toxic to BRCA2 deficient or BRCA2 knockout cells over BRCA2 proficient or BRCA2 wild type cells. In other embodiments, BRCA2 deficient or BRCA2 knockout cells exhibit higher sensitivity to Compound I or a pharmaceutically acceptable salt or solvate thereof or the compound of the present invention as compared to BRCA2 proficient or BRCA2 wild type cells.

In some embodiments, cancer treated or ameliorated by any one of the methods as disclosed herein is characterized by one or more mutations in the BRCA1 or BRCA2 genes. BRCA1 and BRCA2 are tumor suppressor genes, and encode proteins involved in DNA damage repair. Mutations that alter expression or activity of the BRCA1 or BRCA2 proteins may lead to the accumulation of genetic alterations in a cell, and can lead to cancer in a subject. Such mutations are referred to herein as “disease-associated mutations.” In some embodiments, the cancer is characterized one or more mutations in BRCA1 and BRCA2 genes. In some embodiments, the cancer is characterized one or more mutations in BRCA1 gene but has no mutations in BRCA2 gene. In some embodiments, the cancer is characterized one or more mutations in BRCA2 gene but has no mutations in BRCA1 gene.

In some embodiments, cancer treated or ameliorated by any one of the methods as disclosed herein is characterized by one or more disease-associated mutations in BRCA1 or BRCA2. In some embodiments, cancer is characterized by one or more disease-associated mutations in BRCA1 and BRCA2. In some embodiments, cancer is characterized by one or more disease-associated mutations in BRCA1 but harbors no disease-associated mutations in BRCA2. In some embodiments, cancer is characterized by one or more disease-associated mutations in BRCA2 but harbors no disease-associated mutations in BRCA1.

In one embodiment, cancer treated or ameliorated by any one of the methods as disclosed herein is BRCA mutant or BRCA-like mutant cancer. In some embodiments, the BRCA mutant or BRCA-like mutant cancer is a BRCA2-mutated cancer. In other embodiments, the BRCA mutant or BRCA-like mutant cancer is breast cancer, ovarian cancer, pancreatic cancer, or prostate cancer. In one embodiment, the BRCA mutant or BRCA-like mutant cancer is breast cancer or prostate cancer. In one embodiment, cancer treated or ameliorated by any one of the methods as disclosed herein is BRCA mutant cancer. In some embodiments, the BRCA mutant cancer is a BRCA2-mutated cancer. In other embodiments, the BRCA mutant cancer is breast cancer, ovarian cancer, pancreatic cancer, or prostate cancer. In other embodiments, the BRCA mutant cancer is breast cancer, ovarian cancer, or pancreatic cancer. In one embodiment, the BRCA mutant cancer is breast cancer or prostate cancer. In some embodiments, the BRCA2-mutated cancer is breast cancer or ovarian cancer. In some embodiments, the BRCA2-mutated cancer is breast cancer. In some embodiments, the BRCA2-mutated cancer is ovarian cancer.

In some embodiments, cancer treated or ameliorated by any one of the methods as disclosed herein is BRCA-driven cancer. In some embodiments, cancer is BRCA1-driven cancer. In some embodiments, cancer is BRCA2-driven cancer. In some embodiments cancer is BRCA1- and BRCA2-driven cancer. In some embodiments, cancer is neither BRCA1-nor BRCA2-driven cancer.

In one embodiment, the present disclosure relates to a method for treating or ameliorating cell proliferation disorder in a human subject, comprising administering to a subject in need thereof a therapeutically effective amount of a compound of the invention or a formulation prepared from a compound of the present invention as disclosed herein. In some embodiments, the human subject carries a BRCA mutation. In other embodiments, the human subject carries a BRCA2 mutation. In another embodiment, the human subject is homozygous for a mutation in BRCA2.

In one embodiment, the present disclosure relates to a method for treating or ameliorating cell proliferation disorder in a human subject, comprising administering to a subject in need thereof a therapeutically effective amount of a compound of the invention or a formulation prepared from a compound of the present invention. In some embodiments, the human subject carries a BRCA mutation. In other embodiments, the human subject carries a BRCA2 mutation. In another embodiment, the human subject is homozygous for a mutation in BRCA2.

In one embodiment, the BRCA2 mutation causes BRCA2 gene to lose its function. In one embodiment, the BRCA2 mutation is a loss-of-function mutation. In one embodiment, the BRCA2 mutation is substitution, deleterious truncating, splicing, insertion or deletion of BRCA2 gene.

In one embodiment, BRCA2 mutation exists as a coding change or mutation in one or more of 4088insA, c.68-80insT, c.793+34T>G, 999del5, 6503delTT, 4486deG, 2594deC, 5382insC, 3829delT, Q563X, 3438G>T, 1675delA, 999del5, 8295T4A, 9900insA, 5579insA, 7647delTG, 7253delAA, 9303ins31, 3034del4 bp, 5910C3G, 6676insTA, 6085G>T, 8765delAG, 3398delAAAAG, 1499insA, 7525_7526insT, 6174delT, c.289G>T, c.2950G>T, c.7963C>T, c.8878C>T, IVS6p1G4A, 6503-6504delTT, 9132delC, 9254del5, c.9254_9258delATCAT, c.3492_3493insT, 9475A>G, c.9026_9030delATCAT, c.3264insT, c.8978_8991del14, c.156_157insAlu, 6238ins2del21, 10323delCins11, 8876delC, 8138_8142del5, c.8765_8766delAG, exons 21-24 del, c.6589delA, 4817A>G, 8477delAGA, 8984delG, G4X, 3783del10, c.5101C>T, c.5433_5436delGGAA, c.7806-2A>G, c.5291C>G, c.3975_3978dupTGCT, IVS16-2A>G, c.3318C>A, c.4790C>A, 9326insA and 6174deT, 8984delG, 1913T>A, 1342C>A, 3199A>G, 1093A>C, c.3394C>T, c.7697T>C, 5531delTT, C5507G, 6174delT, c.5373_5376 del GTAT, c.373G>T, S2219X, C1290Y, 6633del5, 3034delACAA, 818delA, exons 8-9 del, c.3036_3039delACAA, c.6024_6025_delTA, c.2732_2733insA, c.3870_3873delG, 4150G>T, 6027del4, c.5114_5117delTAAA, c.2639_2640delTG, 6880 insG, 3034 del AAAC, 695insT, 1528del4, 9318del4, S1099X, 5802delAATT, 8732C>A, c.2835C>A, c.7480C>T, 1627A.T, 3972delTGAG, 7708C.T, 7883delTTAA, c.2808_2811delACAA, c.3109C>T, c.7436_7805del370, c.9097_9098insA, 2670delC, 3073delT, 6696-7delTC, exons 4-11 dup, 4859delA, 4265delCT, 1342C.A, 490 delCT, 3337C>T, 5057delTG, g.-1235G>A, g.-26G>A, g.681+56C>T, c.865A>C, c.1114A>C, c.1365A>G, c.2229T>C, c.2971A>G, c.3396A>G, c.3516G>A, c.3807T>C, c.4415_4418delAGAA, c.5529A>C, c.6033_6034insGT, c.7242A>G, g.7435+53C>T, g.7806-14T>C, g.8755-66T>C, c.4415-4418delAGAA, c.6033insGT, c.5576_5579delTTAA, c.9485-1G>A, 4265delCT, 4859delA, 6775G>T, p.Gu2183X, c.2699_2704delTAAATG, 4706delAAAG, R2336P, IVS2+1G>A, 8765delAG, 999 del 5, 1537 del4, 5909 insA, c.211dupA, c.3381delT/3609delT, c.7110delA/7338delA, c.7235insG/7463insG, c.2826_2829del, c.6447_6448dup, c.5771_5774del, and/or 5999del4. See Karami, F. et al. BioMed Res. Int'l. 2013, 2013, Article ID 928562, which is hereby incorporated by reference in its entirety for all purposes.

In one embodiment, BRCA2 mutation exists as a coding change or mutation in one or more of c.8537_8538del AG, c.8537_8538del AG mutation in exon 20, c.859G>C, c. 859G>C in exon 7, c.4614T>C, p.Ser1538Ser synonymous mutation, c.5946delT, p.S1982fs, c.6819DelinsGT, c.6592G>T, c.3847_3848delGT, c.6821G>T, or c.6821G>T in exon 11.

In one embodiment, the compound of the present disclosure demonstrate sensitivity to a BRCA2 null cell line relative to the parental cell line. In one embodiment, the sensitivity of the BRCA2 null cell line is at least two hundred fold greater than the BRCA2 wild type cell line. In other embodiments, the sensitivity is at least twenty fold higher. In some embodiments, the sensitivity is at least 200 fold higher. In other embodiments, the sensitivity is at least 2, 5, 10, 15, 20, 25, 30, 35, 40, 45, 50, 60, 70, 80, 90, 100, 125, 150, 175, 200 or 400 fold higher.

In one embodiment, the present disclosure relates to methods for treating cancer in a subject, comprising administering a therapeutically effective amount of Compound I, or a pharmaceutically acceptable salt and/or solvate thereof to the subject, wherein the subject has a PALB2 mutation and/or a BRCA2 mutation. In one embodiment, the subject has a PALB2 mutation. In one embodiment, the subject has a BRCA2 mutation. In one embodiment, the subject has a PALB2 mutation and a BRCA2 mutation. In one embodiment, the subject has one or more additional gene mutation in the homologous recombination pathway.

In another embodiment, cancer treated or ameliorated by the method comprises cancer cells harboring defects in PALB2 gene. In another embodiment, the cancer cells are deficient in PALB2. In another embodiment, the cancer cells are homozygous for a mutation in PALB2. In another embodiment, the cancer cells are heterozygous for a mutation in PALB2.

In one embodiment, Compound I or a pharmaceutically acceptable salt or solvate thereof or the compound of the present invention induces more apoptotic cell death in PALB2 deficient or PALB2 knockout cells relative to PALB2 proficient or PALB2 wild type cells. In one embodiment, Compound I or a pharmaceutically acceptable salt or solvate thereof or the compound of the present invention is selectively toxic to PALB2 deficient or PALB2 knockout cells over PALB2 proficient or PALB2 wild type cells. In other embodiments, PALB2 deficient or PALB2 knockout cells exhibit higher sensitivity to Compound I or a pharmaceutically acceptable salt or solvate thereof or the compound of the present invention as compared to PALB2 proficient or PALB2 wild type cells.

In some embodiments, cancer treated or ameliorated by any one of the methods as disclosed herein is characterized by one or more mutations in the PALB2 genes. Mutations that alter expression or activity of the PALB2 proteins may lead to the accumulation of genetic alterations in a cell, and can lead to cancer in a subject. Such mutations are referred to herein as “disease-associated mutations.” In some embodiments, the cancer is characterized one or more mutations in PALB2 genes.

In some embodiments, cancer treated or ameliorated by any one of the methods as disclosed herein is characterized by one or more disease-associated mutations in PALB2.

In one embodiment, cancer treated or ameliorated by any one of the methods as disclosed herein is PALB2 mutant or PALB2-like mutant cancer. In some embodiments, the PALB2 mutant or PALB2-like mutant cancer is a PALB2-mutated cancer. In other embodiments, the PALB2 mutant or PALB2-like mutant cancer is breast cancer, ovarian cancer, pancreatic cancer, or prostate cancer. In one embodiment, the PALB2 mutant or PALB2-like mutant cancer is breast cancer or prostate cancer. In one embodiment, cancer treated or ameliorated by any one of the methods as disclosed herein is PALB2 mutant cancer (PALB2-mutated cancer). In other embodiments, the PALB2 mutant cancer is breast cancer, ovarian cancer, pancreatic cancer, or prostate cancer. In other embodiments, the PALB2 mutant cancer is breast cancer, ovarian cancer, or pancreatic cancer. In one embodiment, the PALB2 mutant cancer is breast cancer or prostate cancer. In one embodiment, the PALB2 mutant cancer is breast cancer.

In one embodiment, the PALB2 mutation causes PALB2 gene to lose its function. In one embodiment, the PALB2 mutation is a loss-of-function mutation. In one embodiment, the PALB2 mutation is substitution, deleterious truncating, splicing, insertion or deletion of PALB2 gene. In some embodiments, the PALB2 mutation is monoallelic loss-of-function mutation. In other embodiments, the PALB2 mutation is biallelic loss-of-function mutation.

In one embodiment, the present disclosure relates to a method for treating or ameliorating cell proliferation disorder in a human subject, comprising administering to a subject in need thereof a therapeutically effective amount of a compound of the invention or a formulation prepared from a compound of the present invention as disclosed herein. In some embodiments, the human subject carries a PALB2 mutation. In another embodiment, the human subject is homozygous for a mutation in PALB2.

In some embodiments, cancer treated or ameliorated by any one of the methods as disclosed herein is PALB2-driven cancer.

In one embodiment, the present disclosure relates to a method for treating or ameliorating cell proliferation disorder in a human subject, comprising administering to a subject in need thereof a therapeutically effective amount of a compound of the invention or a formulation prepared from a compound of the present invention. In some embodiments, the human subject carries a PALB2 mutation. In another embodiment, the human subject is homozygous for a mutation in PALB2.

In one embodiment, PALB2 mutation exists as a coding change in one or more of c.48G>A, c.72del, c.156del, c.172_175del, c.196C>T, c.229del, c.451C>T, c.509_510del, c.757_758del, c.886del, c.956_962del, c.1027C>T, c.1037_1041del, c.1108C>T, c.1240C>T, c.1314del, c.1431del, c.1571C>G, c.1591_1600del, c.1592del, c.1653T>A, c.2074C>T, c.2167_2168del, c.2257C>T, c.2323C>T, c.2386G>T, c.2515-1G>T, c.2521del, c.2686dup, c.2718G>A, c.2787_2788del, c.2834+1G>T, c.2835-1G>C, c.2888del, c.2919_2920del, c.2982dup, c.3022del, c.3113G>A, c.3116del, c.3201+1G>C, c.3323del, c.3423_3426del, c.3426dup, c.3456dup, c.3497_3498del, c.3504_3505del, c.3549C>A, c.3549C>G, del5340 bp, or c.3362del. See Antoniou, A. C. et al. N. Engl. J. Med. 2014, 371, 497-506, which is hereby incorporated by reference in its entirety for all purposes.

In one embodiment, the present disclosure relates to methods for treating cancer in a subject, comprising a) determining if the subject harbors a BRCA1, BRCA2, or PALB2 mutation, and b) administering to a subject a therapeutically effective amount of a compound of the invention or a formulation prepared from a compound of the present invention. In one embodiment, the method of treating cancer in a subject comprises a) determining if the subject harbors a BRCA1, BRCA2, or PALB2 mutation, and b) administering to a subject a therapeutically effective amount of a compound of the invention or a formulation prepared from a compound of the present invention if the subject harbors a BRCA1, BRCA2, or PALB2 mutation. In one embodiment, the method of treating cancer in a subject comprises a) determining if the subject harbors BRCA1, BRCA2, or PALB2 mutation, and b) administering to a subject a therapeutically effective amount of a compound of the invention or a formulation prepared from a compound of the present invention if the subject harbors a BRCA2 or PALB2 mutation. In one embodiment, the method of treating cancer in a subject comprises a) determining if the subject harbors BRCA1, BRCA2, or PALB2 mutation, and b) administering to a subject a therapeutically effective amount of a compound of the invention or a formulation prepared from a compound of the present invention if the subject harbors a BRCA2 mutation. In one embodiment, the method of treating cancer in a subject comprises a) determining if the subject harbors BRCA1, BRCA2, or PALB2 mutation, and b) administering to a subject a therapeutically effective amount of a compound of the invention or a formulation prepared from a compound of the present invention if the subject harbors a PALB2 mutation.

In one embodiment, the present disclosure relates to methods for treating cancer in a subject, comprising a) determining if the subject harbors a disease-associated mutation in BRCA1, BRCA2, or PALB2 genes, and b) administering to a subject a therapeutically effective amount of a compound of the invention or a formulation prepared from a compound of the present invention. In one embodiment, the method of treating cancer in a subject comprises a) determining if the subject harbors a disease-associated mutation in BRCA1, BRCA2, or PALB2 genes, and b) administering to a subject a therapeutically effective amount of a compound of the invention or a formulation prepared from a compound of the present invention if the subject harbors a disease-associated mutation in BRCA1, BRCA2, or PALB2 genes. In one embodiment, the method of treating cancer in a subject comprises a) determining if the subject harbors a disease-associated mutation in BRCA1, BRCA2, or PALB2 genes, and b) administering to a subject a therapeutically effective amount of a compound of the invention or a formulation prepared from a compound of the present invention if the subject harbors a disease-associated mutation in BRCA2 or PALB2 genes. In one embodiment, the method of treating cancer in a subject comprises a) determining if the subject harbors a disease-associated mutation in BRCA1, BRCA2, or PALB2 genes, and b) administering to a subject a therapeutically effective amount of a compound of the invention or a formulation prepared from a compound of the present invention if the subject harbors a disease-associated mutation in BRCA2 gene. In one embodiment, the method of treating cancer in a subject comprises a) determining if the subject harbors a disease-associated mutation in BRCA1, BRCA2, or PALB2 genes, and b) administering to a subject a therapeutically effective amount of a compound of the invention or a formulation prepared from a compound of the present invention if the subject harbors a disease-associated mutation in PALB2 gene. In another embodiment, the cancer cells are deficient in BRCA1 and/or BRCA2. In another embodiment, the cancer cells are homozygous for a mutation in BRCA1 and/or BRCA2. In another embodiment, the cancer cells are heterozygous for a mutation in BRCA1 and/or BRCA2. In some embodiments, the cancer cells are deficient in germline BRCA1 and/or BRCA2. In another embodiment, the cancer cells are deficient in somatic BRCA1 and/or BRCA2.

Additionally, the present disclosure relates to methods for treating cancers, cancer cells, tumors, or tumor cells comprising administering a therapeutically effective amount of a compound of the invention or a formulation prepared from a compound of the present invention. The present disclosure also relates to methods for treating cancers, cancer cells, tumors, or tumor cells comprising administering a therapeutically effective amount of a compound of the invention or a formulation prepared from a compound of the present invention, to a subject in need thereof. Non limiting examples of cancer that may be treated by the methods of this disclosure include cancer or cancer cells of: large intestine, breast, ovary, cervix, lung, liver, pancreas, lymph node, colon, rectum, prostate, brain, head and neck, skin, kidney, osteosarcoma, bone (e.g., Ewing's sarcoma), blood and heart (e.g., leukemia, lymphoma, carcinoma), uterine, gastrointestinal malignancies, and carcinomas of the larynx and oral cavity. Non limiting examples of tumors that may be treated by the methods of this disclosure include tumors and tumor cells of: large intestine, breast, ovary, cervix, lung, liver, pancreas, lymph node, colon, rectum, prostate, brain, head and neck, skin, kidney, osteosarcoma, bone (e.g., Ewing's sarcoma), blood and heart (e.g., leukemia, lymphoma, carcinoma), uterine, gastrointestinal malignancies, and carcinomas of the larynx and oral cavity.

The present invention also provides methods of decreasing Pol I transcription comprising administering a compound of the invention or a formulation prepared from a compound of the present invention, to a subject in need. In some embodiments, the inhibition of Pol I transcription is in peripheral blood mononuclear cells (PBMC). In other embodiments, the inhibition of Pol I transcription can be observed in PBMC at one hour post-IV infusion of a dose comprising an effective amount of a compound of the invention or a formulation prepared from a compound of the present invention.

In one embodiment, the inhibition of Pol I transcription in PBMC 1 hour post-infusion is at an average level of about 15% inhibition or greater. In another embodiment, the Pol I transcription in PBMC 1 hour post-infusion is at an average level of about 5% inhibition or greater, about 10% inhibition or greater, about 15% inhibition or greater, about 20% inhibition or greater, about 25% inhibition or greater, about 30% inhibition or greater, about 35% inhibition or greater, about 40% inhibition or greater, about 45% inhibition or greater, about 50% inhibition or greater, about 55% inhibition or greater, about 65% inhibition or greater, or about 70% inhibition or greater.

In one embodiment of the present methods disclosed herein, the inhibition of Pol I transcription can be observed in MACS (magnetic-activated cell sorting) sorted tumor cells.

As used herein, administering can be effected or performed using any of the various methods known to those skilled in the art. A compound of the invention or a formulation prepared from a compound of the present invention, can be administered, for example, subcutaneously, intravenously, parenterally, intraperitoneally, intradermally, intramuscularly, topically, enteral (e.g., orally), rectally, nasally, buccally, sublingually, vaginally, by inhalation spray, by drug pump or via an implanted reservoir in dosage formulations containing conventional non-toxic, physiologically acceptable carriers or vehicles. A formulation or a composition comprising the compound of the present invention can be administered, for example, subcutaneously, intravenously, parenterally, intraperitoneally, intradermally, intramuscularly, topically, enteral (e.g., orally), rectally, nasally, buccally, sublingually, vaginally, by inhalation spray, by drug pump or via an implanted reservoir in dosage formulations containing conventional non-toxic, physiologically acceptable carriers or vehicles. In one embodiment, the composition of the present disclosure is administered intravenously.

Further, a compound of the invention or a formulation prepared from a compound of the present invention, can be administered to a localized area in need of treatment. For example, a formulation prepared from a compound of the present invention can be administered to a localized area in need of treatment. Administration to a localized area can be achieved by, for example, and not by way of limitation, local infusion during surgery, topical application, transdermal patches, by injection, by catheter, by suppository, or by implant (the implant can optionally be of a porous, non-porous, or gelatinous material), including membranes, such as sialastic membranes or fibers.

The formulation of a compound of the present invention for administration (e.g., syrup, elixir, capsule, tablet, foams, emulsion, gel, etc.) to a subject will depend in part on the route by which it is administered. For example, for mucosal (e.g., oral mucosa, rectal, intestinal mucosa, bronchial mucosa) administration, nose drops, aerosols, inhalants, nebulizers, eye drops or suppositories can be used. A compound of the invention or a formulation prepared from a compound of the present invention can also be used to coat bioimplantable materials to enhance neurite outgrowth, neural survival, or cellular interaction with the implant surface. A compound of the invention or a formulation prepared from a compound of the present invention can be administered together with other biologically active agents, such as anticancer agents, analgesics, anti-inflammatory agents, anesthetics and other agents which can control one or more symptoms or causes of a disorder or a condition characterized by cell proliferation.

In one embodiment, a compound of the invention or a formulation prepared from a compound of the present invention, as disclosed herein, can be administered in combination with one or more therapeutically active agent. In one embodiment, the one or more therapeutically active agent is an anticancer agent. In some embodiments, the one or more therapeutically active anticancer agents include, but are not limited to, paclitaxel, vinblastine, vincristine, etoposide, doxorubicin, hercepztin, lapatinib, gefitinib, erlotinib, tamoxifen, fulvestrant, anastrazole, lectrozole, exemestane, fadrozole, cyclophosphamide, taxotere, melphalan, chlorambucil, mechlorethamine, chlorambucil, phenylalanine, mustard, cyclophosphamide, ifosfamide, carmustine (BCNU), lomustine (CCNU), streptozotocin, busulfan, thiotepa, cisplatin, carboplatin, dactinomycin (actinomycin D), doxorubici(adriamycin), daunorubicin, idarubicin, mitoxantrone, plicamycin, mitomycin C, bleomycin, combinations thereof, and the like. In another embodiment, the one or more therapeutically active anticancer agents include, but are not limited to, PARP (poly (DP-ribose)polymerase) inhibitors. Suitable PARP inhibitors include, but are not limited to, 4-(3-(1-(cyclopropanecarbonyl)piperazine-4-carbonyl)-4-fluorobenzyl)phthalazin-1(2H)-one (olaparib, AZD2281, Ku-0059436), 2-[(2R)-2-methylpyrrolidin-2-yl]-1H-benzimidazole-4-carboxamide (Veliparib, ABT-888), (8S,9R)-5-fluoro-8-(4-fluorophenyl)-9-(1-methyl-1H-1,2,4-triazol-5-yl)-8,9-dihydro-2H-pyrido[4,3,2-de]phthalazin-3(7H)-one (talazoparib, BMN 673), 4-iodo-3-nitrobenzamide (iniparib, BSI-201), 8-fluoro-5-(4-((methylamino)methyl)phenyl)-3,4-dihydro-2H-azepino[5,4,3-cd]indol-1(6H)-one phosphoric acid (Rucaparib, AG-014699, PF-01367338), 2-[4-[(dimethylamino)methyl]phenyl]-5,6-dihydroimidazo[4,5,1-jk][1,4]benzodiazepin-7(4H)-one (AG14361), 3-aminobenzamide (INO-1001), 2-(2-fluoro-4-((S)-pyrrolidin-2-yl)phenyl)-3H-benzo[d]imidazole-4-carboxamide (A-966492), N-(5,6-dihydro-6-oxo-2-phenanthridinyl)-2-acetamide hydrochloride (PJ34, PJ34 HCl), MK-4827, 3,4-dihydro-4-oxo-3,4-dihydro-4-oxo-N-[(1S)-1-phenylethyl]-2-quinazolinepropanamide (ME0328), 5-(2-oxo-2-phenylethoxy)-1(2H)-isoquinolinone (UPF-1069), 4-[[4-fluoro-3-[(4-methoxy-1-piperidinyl)carbonyl]phenyl]methyl]-1(2H)-phthalazinone (AZD 2461), 5-((3-chlorophenyl)amino)benzo[c][2,6]naphthyridine-8-carboxylic acid, and the like. In another embodiment, the one or more therapeutically active agent is an immunotherapeutic agent. In some embodiments, the one or more immunotherapeutic agents includes, but are not limited to, a monoclonal antibody, an immune effector cell, adoptive cell transfer, an immunotoxin, a vaccine, a cytokine, and the like.

In one embodiment, the one or more therapeutically active agent is selected from an alkylating agent, an anti-metabolite, a vinca alkaloid, a taxane, a topoisomerase inhibitor, an anti-tumor antibiotic, a tyrosine kinase inhibitor, an immunosuppressive macrolide, an Akt inhibitor, an HDAC inhibitor an Hsp90 inhibitor, an mTOR inhibitor, a PI3K/mTOR inhibitor, a PI3K inhibitor, a CDK (cyclin-dependent kinase) inhibitor, CHK (checkpoint kinase) inhibitor, PARP (poly (DP-ribose)polymerase) inhibitors, or combinations thereof.

In one embodiment, the one or more therapeutically active agent is a PI3K inhibitor. In another embodiment, the PI3K inhibitor is Idelalisib.

In one embodiment, the one or more therapeutically active agent is a PARP inhibitor. In another embodiment, the PARP inhibitor is Olaparib.

In other embodiments, the one or more therapeutically active agent is an agent that induces immune checkpoint blockade, such as PD-1 blockade and CTLA-4 blockade.

In some embodiments, the one or more therapeutically active agent is an antibody or an antigen-binding portion thereof that disrupts the interaction between Programmed Death-1 (PD-1) and Programmed Death Ligand-1 (PD-L1). In one embodiment, the one or more therapeutically active agent is selected from the group consisting of an anti-PD-1 antibody, a PD-1 antagonist, an anti-PD-L1 antibody, a siRNA targeting expression of PD-1, a siRNA targeting the expression of PD-L1, and a peptide, fragment, dominant negative form, or soluble form of PD-1 or PD-L1.

In one embodiment, the one or more therapeutically active agent is a monoclonal antibody. In one embodiment, the monoclonal antibody is selected from the group consisting of anti-PD-1 antibody, nivolumab, pembrolizumab alemtuzumab, bevacizumab, brentuxima b vedotin, cetuximab, gemtuzumab ozogamicin, ibritumomab tiuxetan, ipilimumab, ofatumumab, panitumumab, rituximab, tositumomab, trastuzumab, anti-B7-H4, anti-B7-H1, anti-LAG3, BTLA, anti-Tim3, anti-B7-DC, anti-CD160, MR antagonist antibodies, anti-4-1BB, anti-OX40, anti-CD27, and/or CD40 agonist antibodies. In some embodiments, the one or more therapeutically active agent is an anti-PD-1 antibody. In other embodiments, an anti-PD-1 antibody is a humanized antibody. In one embodiment, the monoclonal antibody is selected from the group consisting of nivolumab and pembrolizumab. In a specific embodiment, the monoclonal antibody is nivolumab.

In some embodiments, one or more therapeutically active agent disclosed in WO 2017/087235 is hereby incorporated by reference in its entirety for all purposes.

In another embodiment, the crystalline form of Compound I, or the the crystalline form of pharmaceutically acceptable salt, ester, and/or solvate of Compound I, as disclosed herein, can be administered in combination with radiotherapy.

Additionally, administration can comprise administering to the subject a plurality of dosages over a suitable period of time. Such administration regimens can be determined according to routine methods, upon a review of the instant disclosure.

Crystalline forms of the invention are generally administered in a dose of about 0.01 mg/kg/dose to about 100 mg/kg/dose. Alternately the dose can be from about 0.1 mg/kg/dose to about 10 mg/kg/dose; or about 1 mg/kg/dose to 10 mg/kg/dose. Time release preparations may be employed or the dose may be administered in as many divided doses as is convenient. When other methods are used (e.g. intravenous administration), crystalline forms are administered to the affected tissue at a rate from about 0.05 to about 10 mg/kg/hour, alternately from about 0.1 to about 1 mg/kg/hour. Such rates are easily maintained when these crystalline forms are intravenously administered as discussed herein. Generally, topically administered formulations are administered in a dose of about 0.5 mg/kg/dose to about 10 mg/kg/dose range. Alternately, topical formulations are administered at a dose of about 1 mg/kg/dose to about 7.5 mg/kg/dose or even about 1 mg/kg/dose to about 5 mg/kg/dose.

A range of from about 0.1 to about 100 mg/kg is appropriate for a single dose. Continuous administration is appropriate in the range of about 0.05 to about 10 mg/kg.

Drug doses can also be given in milligrams per square meter of body surface area rather than body weight, as this method achieves a good correlation to certain metabolic and excretionary functions. Moreover, body surface area can be used as a common denominator for drug dosage in adults and children as well as in different animal species (Freireich et al., (1966) Cancer Chemother Rep. 50, 219-244). Briefly, to express a mg/kg dose in any given species as the equivalent mg/sq m dose, the dosage is multiplied by the appropriate km factor. In an adult human, 100 mg/kg is equivalent to 100 mg/kg×37 kg/sq m=3700 mg/m².

A dosage form of the present invention may contain Compound I, or a pharmaceutically acceptable salt, ester, and/or solvate thereof, as disclosed herein, in an amount of about 5 mg to about 500 mg. That is, a dosage form of the present invention may contain Compound I in an amount of about 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 125 mg, 130 mg, 140 mg, 150 mg, 160 mg, 170 mg, 175 mg, 180 mg, 190 mg, 200 mg, 210 mg, 220 mg, 225 mg, 230 mg, 240 mg, 250 mg, 260 mg, 270 mg, 275 mg, 280 mg, 290 mg, 300 mg, 310 mg, 320 mg, 325 mg, 330 mg, 340 mg, 350 mg, 360 mg, 370 mg, 375 mg, 380 mg, 390 mg, 400 mg, 410 mg, 420 mg, 425 mg, 430 mg, 440 mg, 450 mg, 460 mg, 470 mg, 475 mg, 480 mg, 490 mg, 500 mg or any value in between. In one embodiment, such dosage amount is administered to a patient as a daily dose either in a single dose or in divided portions served multiple times a day, such as twice, three times, or four times a day.

In one embodiment, compounds of the present invention or formulation prepared by compounds of the present invention are generally administered in a dose of about 1 mg/m² to about 2000 mg/m², or any value or subranges therebetween, of Compound I, or a pharmaceutically acceptable salt and/or solvate thereof. In one embodiment, compounds of the present invention or formulation prepared by compounds of the present invention are administered in a dose of about 10 mg/m² to about 1500 mg/m², or any value or subranges therebetween, of Compound I, or a pharmaceutically acceptable salt and/or solvate thereof. In another embodiment, compounds of the present invention or formulation prepared by compounds of the present invention are administered in a dose of about 200 mg/m² to about 800 mg/m², or any value or subranges therebetween, of Compound I, or a pharmaceutically acceptable salt and/or solvate thereof. In another embodiment, compounds of the present invention or formulation prepared by compounds of the present invention are administered in a dose of about 20 mg/m² to about 300 mg/m², or any value or subranges therebetween, of Compound I, or a pharmaceutically acceptable salt and/or solvate thereof. In some embodiments, the dose can vary dependent on the type of diseases or conditions which Compound I, or a pharmaceutically acceptable salt and/or solvate thereof is being administered for (e.g., cancer or solid tumor). In some embodiments, the dose can vary depending on the health of the patients or the patient's sensitivity to Compound I, or a pharmaceutically acceptable salt and/or solvate thereof.

In one embodiment, the compounds of the present invention or formulation prepared by compounds of the present invention are administered in a dose of about 25 mg/m² to about 2000 mg/m², or any value or subranges therebetween, of Compound I, or a pharmaceutically acceptable salt and/or solvate thereof. In one embodiment, compounds of the present invention or formulation prepared by compounds of the present invention can be administered in a dose of about 25 mg/m², about 30 mg/m², about 35 mg/m², about 40 mg/m², about 45 mg/m², about 50 mg/m², about 55 mg/m², about 60 mg/m², about 65 mg/m², about 70 mg/m², about 75 mg/m², about 80 mg/m², about 85 mg/m², about 90 mg/m², about 95 mg/m², about 100 mg/m², about 110 mg/m², about 120 mg/m², about 125 mg/m², about 130 mg/m², about 140 mg/m², about 150 mg/m², about 160 mg/m², about 170 mg/m², about 175 mg/m², about 180 mg/m², about 190 mg/m², about 200 mg/m², about 210 mg/m², about 220 mg/m², about 225 mg/m², about 230 mg/m², about 240 mg/m², about 250 mg/m², about 260 mg/m², about 270 mg/m², about 275 mg/m², about 280 mg/m², about 290 mg/m², about 300 mg/m², about 310 mg/m², about 320 mg/m², about 325 mg/m², about 330 mg/m², about 340 mg/m², about 350 mg/m², about 360 mg/m², about 370 mg/m², about 375 mg/m², about 380 mg/m², about 390 mg/m², about 400 mg/m², about 410 mg/m², about 420 mg/m², about 425 mg/m², about 430 mg/m², about 440 mg/m², about 450 mg/m², about 460 mg/m², about 470 mg/m², about 475 mg/m², about 480 mg/m², about 490 mg/m², about 500 mg/m², about 510 mg/m², about 520 mg/m², about 525 mg/m², about 530 mg/m², about 540 mg/m², about 550 mg/m², about 560 mg/m², about 570 mg/m², about 575 mg/m², about 580 mg/m², about 590 mg/m², about 600 mg/m², about 610 mg/m², about 620 mg/m², about 625 mg/m², about 630 mg/m², about 640 mg/m², about 650 mg/m², about 660 mg/m², about 670 mg/m², about 675 mg/m², about 680 mg/m², about 690 mg/m², about 700 mg/m², about 710 mg/m², about 720 mg/m², about 725 mg/m², about 730 mg/m², about 740 mg/m², about 750 mg/m², about 760 mg/m², about 770 mg/m², about 775 mg/m², about 780 mg/m², about 790 mg/m², about 800 mg/m², about 810 mg/m², about 820 mg/m², about 825 mg/m², about 830 mg/m², about 840 mg/m², about 850 mg/m², about 860 mg/m², about 870 mg/m², about 875 mg/m², about 880 mg/m², about 890 mg/m², about 900 mg/m², about 910 mg/m², about 920 mg/m², about 925 mg/m², about 930 mg/m², about 940 mg/m², about 950 mg/m², about 960 mg/m², about 970 mg/m², about 975 mg/m², about 980 mg/m², about 990 mg/m², about 1000 mg/m², about 1010 mg/m², about 1020 mg/m², about 1025 mg/m², about 1030 mg/m², about 1040 mg/m², about 1050 mg/m², about 1060 mg/m², about 1070 mg/m², about 1075 mg/m², about 1080 mg/m², about 1090 mg/m², about 1100 mg/m², about 1110 mg/m², about 1120 mg/m², about 1125 mg/m², about 1130 mg/m², about 1140 mg/m², about 1150 mg/m², about 1160 mg/m², about 1170 mg/m², about 1175 mg/m², about 1180 mg/m², about 1190 mg/m², about 1200 mg/m², about 1210 mg/m², about 1220 mg/m², about 1225 mg/m², about 1230 mg/m², about 1240 mg/m², about 1250 mg/m², about 1260 mg/m², about 1270 mg/m², about 1275 mg/m², about 1280 mg/m², about 1290 mg/m², about 1300 mg/m², about 1310 mg/m², about 1320 mg/m², about 1325 mg/m², about 1330 mg/m², about 1340 mg/m², about 1350 mg/m², about 1360 mg/m², about 1370 mg/m², about 1375 mg/m², about 1380 mg/m², about 1390 mg/m², about 1400 mg/m², about 1410 mg/m², about 1420 mg/m², about 1425 mg/m², about 1430 mg/m², about 1440 mg/m², about 1450 mg/m², about 1460 mg/m², about 1470 mg/m², about 1475 mg/m², about 1480 mg/m², about 1490 mg/m², about 1500 mg/m², about 1510 mg/m², about 1520 mg/m², about 1525 mg/m², about 1530 mg/m², about 1540 mg/m², about 1550 mg/m², about 1560 mg/m², about 1570 mg/m², about 1575 mg/m², about 1580 mg/m², about 1590 mg/m², about 1500 mg/m², about 1610 mg/m², about 1620 mg/m², about 1625 mg/m², about 1630 mg/m², about 1640 mg/m², about 1650 mg/m², about 1660 mg/m², about 1670 mg/m², about 1675 mg/m², about 1680 mg/m², about 1690 mg/m², about 1700 mg/m², about 1710 mg/m², about 1720 mg/m², about 1725 mg/m², about 1730 mg/m², about 1740 mg/m², about 1750 mg/m², about 1760 mg/m², about 1770 mg/m², about 1775 mg/m², about 1780 mg/m², about 1790 mg/m², about 1800 mg/m², about 1810 mg/m², about 1820 mg/m², about 1825 mg/m², about 1830 mg/m², about 1840 mg/m², about 1850 mg/m², about 1860 mg/m², about 1870 mg/m², about 1875 mg/m², about 1880 mg/m², about 1890 mg/m², about 1900 mg/m², about 1910 mg/m², about 1920 mg/m², about 1925 mg/m², about 1930 mg/m², about 1940 mg/m², about 1950 mg/m², about 1960 mg/m², about 1970 mg/m², about 1975 mg/m², about 1980 mg/m², about 1990 mg/m², about 2000 mg/m², or any value in between, of Compound I, or a pharmaceutically acceptable salt and/or solvate thereof.

In one embodiment, compounds of the present invention or formulation prepared by compounds of the present invention are administered in a dose of about 150 mg/m² to about 700 mg/m², or any value or subranges therebetween, of Compound I, or a pharmaceutically acceptable salt and/or solvate thereof. In one embodiment, compounds of the present invention or formulation prepared by compounds of the present invention are administered in a dose of about 150 mg/m² to about 300 mg/m², or any value or subranges therebetween, of Compound I, or a pharmaceutically acceptable salt and/or solvate thereof. In one embodiment, compounds of the present invention or formulation prepared by compounds of the present invention are administered in a dose of about 150 mg/m² to about 250 mg/m², or any value or subranges therebetween, of Compound I, or a pharmaceutically acceptable salt and/or solvate thereof. In one embodiment, compounds of the present invention or formulation prepared by compounds of the present invention are administered in a dose of about 170 mg/m² of Compound I, or a pharmaceutically acceptable salt and/or solvate thereof.

In one embodiment, compounds of the present invention or formulation prepared by compounds of the present invention are administered in a dose of about 300 mg/m² to about 700 mg/m², or any value or subranges therebetween, of Compound I, or a pharmaceutically acceptable salt and/or solvate thereof. In one embodiment, compounds of the present invention or formulation prepared by compounds of the present invention are administered in a dose of about 400 mg/m² to about 700 mg/m², or any value or subranges therebetween, of Compound I, or a pharmaceutically acceptable salt and/or solvate thereof. In one embodiment, compounds of the present invention or formulation prepared by compounds of the present invention are administered in a dose of about 425 mg/m² to about 675 mg/m², or any value or subranges therebetween, of Compound I, or a pharmaceutically acceptable salt and/or solvate thereof. In one embodiment, compounds of the present invention or formulation prepared by compounds of the present invention are administered in a dose of about 450 mg/m² to about 650 mg/m², or any value or subranges therebetween, of Compound I, or a pharmaceutically acceptable salt and/or solvate thereof. In one embodiment, compounds of the present invention or formulation prepared by compounds of the present invention are administered in a dose of about 475 mg/m² of Compound I, or a pharmaceutically acceptable salt and/or solvate thereof.

In one embodiment, compounds of the present invention or formulation prepared by compounds of the present invention can be generally administered in a dose of about less than about 500 mg/m² of Compound I, or a pharmaceutically acceptable salt and/or solvate thereof. In another embodiment, compounds of the present invention or formulation prepared by compounds of the present invention are generally administered in a dose of less than about 500 mg/m², less than about 490 mg/m², less than about 480 mg/m², less than about 475 mg/m², less than about 470 mg/m², less than about 460 mg/m², less than about 450 mg/m², less than about 440 mg/m², less than about 430 mg/m², less than about 420 mg/m², less than about 410 mg/m², less than about 400 mg/m², less than about 390 mg/m², less than about 380 mg/m², less than about 375 mg/m², less than about 370 mg/m², less than about 360 mg/m², less than about 350 mg/m², less than about 340 mg/m², less than about 330 mg/m², less than about 320 mg/m², less than about 310 mg/m², less than about 300 mg/m², less than about 290 mg/m², less than about 280 mg/m², less than about 275 mg/m², less than about 270 mg/m², less than about 260 mg/m², less than about 250 mg/m², less than about 240 mg/m², less than about 230 mg/m², less than about 220 mg/m², less than about 210 mg/m², less than about 200 mg/m², less than about 190 mg/m², less than about 180 mg/m², or less than about 170 mg/m², or any value in between, of Compound I, or a pharmaceutically acceptable salt and/or solvate thereof.

In some embodiments, compounds of the present invention or formulation prepared by compounds of the present invention can be administered to a cancer patient in a dose of less than about 750 mg/m², less than about 700 mg/m², less than about 600 mg/m², less than about 500 mg/m², less than about 475 mg/m², less than about 400 mg/m², less than about 325 mg/m², less than about 300 mg/m², less than about 200 mg/m², less than about 170 mg/m², or any subranges therein, of Compound I, or a pharmaceutically acceptable salt and/or solvate thereof. In other embodiments, compounds of the present invention or formulation prepared by compounds of the present invention can be administered to a cancer patient in a dose of less than about 170 mg/m² of Compound I, or a pharmaceutically acceptable salt and/or solvate thereof, every three weeks. In one embodiment, the cancer patient is a heme cancer patient.

In some embodiments, compounds of the present invention or formulation prepared by compounds of the present invention can be administered to a cancer patient in about 50 mg/m² to about 1550 mg/m², about 150 mg/m² to about 1250 mg/m², about 250 mg/m² to about 1050 mg/m², about 350 mg/m² to about 950 mg/m², about 375 mg/m² to about 850 mg/m², about 425 mg/m² to about 850 mg/m², about 450 mg/m² to about 800 mg/m², or about 500 mg/m² to about 750 mg/m², or any subranges therein, of Compound I, or a pharmaceutically acceptable salt and/or solvate thereof. In some embodiments, compounds of the present invention or formulation prepared by compounds of the present invention can be administered to a cancer patient in a dose of less than about 750 mg/m² of Compound I, or a pharmaceutically acceptable salt and/or solvate thereof. In other embodiments, compounds of the present invention or formulation prepared by compounds of the present invention can be administered to a cancer patient in any of the dosing frequency, dosing cycle or dosing regimen described herein. In one embodiment, the treatment is for solid tumors.

A dosage form of the present invention may be administered, hourly, daily, weekly, or monthly. The dosage form of the present invention may be administered twice a day or once a day. The dosage form of the present invention may be administered with food or without food.

In one embodiment, compounds of the present invention or formulation prepared by compounds of the present invention, is administered once a week, once every two weeks, once every three weeks, once every four weeks, or once a month. In some embodiments, compounds of the present invention or formulation prepared by compounds of the present invention, is administered in a four-week treatment cycle comprising one administration weekly (QW×4). In some embodiments, compounds of the present invention or formulation prepared by compounds of the present invention, is administered in a four-week treatment cycle comprising one administration weekly for two weeks followed by two weeks of rest period (no treatment) (QW×2). In some embodiments, the administration is on a four-week treatment cycle comprising one administration weekly for three weeks followed by one week of rest period (no treatment). In some embodiments, compounds of the present invention or formulation prepared by compounds of the present invention, is administered in a three-week treatment cycle comprising one administration weekly for two weeks followed by one week of rest period. In another embodiment, compounds of the present invention or formulation prepared by compounds of the present invention, is administered once every three weeks. In other embodiments, compounds of the present invention or formulation prepared by compounds of the present invention, is administered once every three weeks by IV infusion.

In some embodiments, the treatment regimen with Compound I, or a pharmaceutically acceptable salt and/or solvate thereof, as disclosed herein, can last from 1 cycle to 20 cycles or greater period of time. An appropriate length of the treatment can be determined by a physician.

In some embodiments, the treatment with the compound of the invention results in PK ranges as disclosed in PCT/US2019/018225, the disclosures of which are hereby incorporated by reference in their entireties for all purposes.

Insofar as the crystalline forms disclosed herein can take the form of a mimetic or fragment thereof, it is to be appreciated that the potency, and therefore dosage of an effective amount can vary. However, one skilled in the art can readily assess the potency of a crystalline form of the type presently envisioned by the present application.

In settings of a gradually progressive disorder or condition characterized by cell proliferation, crystalline forms of the present application are generally administered on an ongoing basis. In certain settings administration of a crystalline form disclosed herein can commence prior to the development of disease symptoms as part of a strategy to delay or prevent the disease. In other settings a crystalline form disclosed herein is administered after the onset of disease symptoms as part of a strategy to slow or reverse the disease process and/or part of a strategy to improve cellular function and reduce symptoms.

It will be appreciated by one of skill in the art that dosage range will depend on the particular crystalline form, and its potency. The dosage range is understood to be large enough to produce the desired effect in which the neurodegenerative or other disorder and the symptoms associated therewith are ameliorated and/or survival of the cells is achieved, but not be so large as to cause unmanageable adverse side effects. It will be understood, however, that the specific dose level for any particular patient will depend on a variety of factors including the activity of the specific crystalline form employed; the age, body weight, general health, sex and diet of the individual being treated; the time and route of administration; the rate of excretion; other drugs which have previously been administered; and the severity of the particular disease undergoing therapy, as is well understood by those skilled in the art. The dosage can also be adjusted by the individual physician in the event of any complication. No unacceptable toxicological effects are expected when crystalline forms disclosed herein are used in accordance with the present application.

An effective amount of the crystalline forms disclosed herein comprise amounts sufficient to produce a measurable biological response. Actual dosage levels of active ingredients in a therapeutic crystalline form of the present application can be varied so as to administer an amount of the active crystalline form that is effective to achieve the desired therapeutic response for a particular subject and/or application. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

Further with respect to the methods of the present application, a preferred subject is a vertebrate subject. A preferred vertebrate is warm-blooded; a preferred warm-blooded vertebrate is a mammal. The subject treated by the presently disclosed methods is desirably a human, although it is to be understood that the principles of the present application indicate effectiveness with respect to all vertebrate species which are included in the term “subject.” In this context, a vertebrate is understood to be any vertebrate species in which treatment of a neurodegenerative disorder is desirable.

As such, the present application provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos or farms. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economical importance to humans. Thus, also provided are the treatment of livestock, including, but not limited to, domesticated swine, ruminants, ungulates, horses (including race horses), poultry, and the like.

The following examples further illustrate the present invention but should not be construed as in any way limiting its scope.

### EXAMPLES

Analytical Methods—various analytical methods, as described below, were applied to the present crystalline forms and their precursors to characterize their physiochemical properties.

Differential Scanning Calorimetry (DSC):

DSC data were collected a TA instrument MDSC Q200. In general, samples in the mass range of 1 to 5 mg were loaded onto a T-zero hermetic pan with a pinhole in the lid and the analysis was carried out under constant flow of nitrogen (60 mL/min). The heating process was programmed to start from 30° C. and stop at 300° C. with a 10° C./min ramp.

Thermogravimetric Analysis (TGA):

In general, samples were placed in a lean and dry aluminum oxide pan or an aluminum pan. Generally, the sample pan and scanned between 20° C. to about 300° C. at 10° C./minute using a nitrogen purge flow rate at about 50 mL/min.

X-Ray Powder Diffraction (XRPD):

XRPD patterns were collected on Bruker AXS D8 diffractometer using Cu Kα₁ radiation (40 kV, 40 mA), θ-2θ goniometer, and divergence of 10 mm slits, a Ge monochromator and LynxEye detector. The representative XRPD pattern was collected under ambient condition. The scanning parameters are: angular range of 5-40°, step size of 0.02°, and scan speed of 0.6 sec/step.

### Example 1. Preparation of Compound I Polymorph J (Free Base)

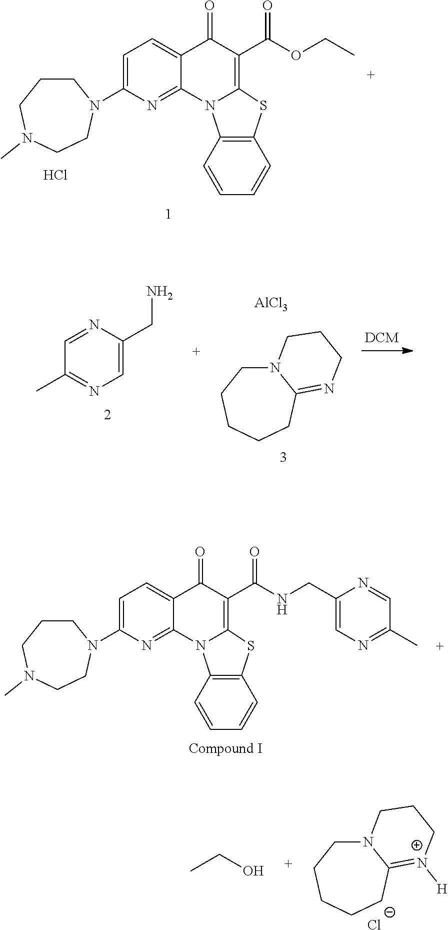

Amidation (crude Compound I): 49.00 kg of dichloromethane (DCM) and 2.100 kg of Compound 1 were charged into a reactor followed by 2.00 kg of DCM rinse. The resulting mixture was stirred at not more than 30° C. for no longer than 10 minutes. Then 2.690 kg of Compound 3 was added at no more than 30° C. then the resulting mixture was stirred at no more than 30° C. for no longer than 10 minutes. The mixture was transferred to another reactor followed by 4.00 kg DCM rinse. To that reactor, 1.120 kg of Compound 2 was charged at no more than 30° C. Then the resulting mixture was cooled down to −5° C. to 5° C. To that, 0.710 kg of AlCl₃ was slowly charged while maintaining the temperature at no more than 10° C. After complete addition of AlCl₃, the reaction mixture was adjusted to 0° C. to 10° C. and stirred at 0° C. to 10° C. for no longer than 2 hours.

Upon the reaction completion, the reaction mixture was transferred into a different reactor followed by 10.00 kg of DCM rinse. To that, 24.950 kg of 6.4% NaOH (aq) was fed through a flow meter while maintaining the temperature at no more than 10° C. Then the reaction mixture was adjusted to 20° C. to 30° C. and stirred at 20° C. to 30° C. for no longer than 30 minutes. Then, 0.600 kg of acid wash celite 545 was charged into the reactor and the reaction mixture was stirred at 20° C. to 30° C. for no longer than 10 minutes. The reaction mixture was transferred through a filter into another reactor and rinsed with 5.55 kg of DCM.

The reaction mixture was stirred and settled for separation. The lower (organic) layer was transferred to another vessel and the upper (aqueous) layer and emulsion layer was left in the reactor. 11.10 kg of DCM was charged into the reactor containing the aqueous layer. The solution was stirred and settled for phase separation. The organic layer was transferred to the vessel containing the organic layer from the first separation. The aqueous layer was discarded.

The combined organic layer was transferred to a reactor and charged with 12.300 kg of 10% NaCl (aq). The solution was stirred and settled, then separated. The separated organic layer was placed under vacuum with temperature controlled at no more than 30° C. until 11 L remained. 25.00 Kg of methanol was charged to the organic layer and the resulting mixture was distilled under vacuum at no more than 30° C. until 17 L remained. The distillate was collected as waste. After distillation was completed, 3.00 kg of methanol was rinsed into the reactor and the resulting slurry was stirred at 20° C. to 30° C. for no longer than 2 hours.

The slurry was filtered and the wet cake, crude Compound I, was washed with 50.00 kg of methanol.

4.80 kg of hydrochloric acid (HCl) and 9.85 kg of purified process water (PPW) were charged into a reactor then 3.003 kg of crude Compound I was charged and stirred at 20° C. to 30° C. for no longer than 90 min. The mixture was filtered and 33.20 kg of methanol was charged and the resulting mixture was filtered. 7.00 kg of 30% NaOH (aq) was added to the reaction mixture while maintaining the temperature at 20° C. to 30° C. until pH was not less than 5. The pH adjusted mixture was transferred to a different reactor and 0.05 kg of 30% NaOH (aq) was added to the reaction mixture while maintaining the temperature at 20° C. to 30° C. until pH was 12-13. Once the pH was adjusted to 12-13, the mixture was heated to 60 to 65° C. and stirred for no longer than 4 hours and then cooled down to 20° C. to 30° C. followed by stirring for no longer than 1 hour. The slurry was filtered and the wet cake was washed with 7.20 kg of MeOH/PPW and then washed with 18.00 kg of PPW until pH of wash liquor reached about 7. The wet cake (final crude Compound I), 2.073 kg, was washed with 3.60 kg of MeOH/PPW and the wet cake was vacuum dried at no more than 65° C.

HCl salt formation: 25.00 kg of DCM and 1.713 kg of final crude Compound I was charged into a reactor. The reaction mixture was transferred to another reactor followed by 43.00 kg of DCM rinse. The resulting mixture was stirred at 20° C. to 30° C. until fully dissolved. In a separate vessel, 0.40 kg of hydrochloric acid (min. 32%) and 2.7 kg of methanol were charged and the mixture was transferred to the reactor containing Compound I, slowly for no longer than 1 hour while maintaining temperature at no more than 30° C. The resulting mixture was stirred at 20° C. to 30° C. for no longer than 2 hours.

The reaction mixture was filtered and washed with 14.00 kg of DCM. The wet cake, Compound I HCl, was dried under vacuum at no more than 40° C. until its loss on drying (LOD) reached no more than 2.0%.

Neutralization/Polymorph Formation: 4.10 kg of HCl and 8.00 kg of PPW were charged into a reactor, then 1.726 kg Compound I HCl salt was added and stirred at 20° C. to 30° C. for no longer than 90 minutes. The reaction was transferred to another reactor and the previous reactor was washed with 29.00 kg methanol, which was added to the new reactor. 6.05 kg of 30% NaOH (aq) was fed into the reactor containing Compound I HCl while the temperature was maintained at 20° C. to 30° C. until pH became no less than 5. Then the mixture was transferred to another reactor and 0.07 kg of 30% NaOH (aq) was added while maintaining the temperature at 20° C. to 30° C. until pH 12-13. Crystal forms precipitated out around pH 9-10. At the scale conducted, it took about 4 hours to adjust the pH to 12-13. The mixture was then heated to 60° C. to 65° C. and stirred for no longer than 4 hours and then cooled down to 20° C. to 30° C. and stirred for no longer than 1 hour. The resulting slurry was filtered and the wet cake was washed with 5.80 kg of MeOH/PPW (3:1) and then washed with 84.00 kg of PPW until pH of wash liquor reached about 7. Finally the wet cake was washed with 3.40 kg of MeOH/PPW and the wet cake was vacuum dried at no more than 65° C. until the water content was not more than 0.5% (Karl Fisher analysis), the methanol content was not more than 2400 ppm, DCM was not more than 600 ppm to provide crystalline Polymorph J (1.4821 kg, 66% yield, >98% chemical purity). The obtained Polymorph J exhibited an XRPD pattern as shown in FIG. 1, a DSC thermogram as shown in FIG. 3 (top line), a TGA thermogram as shown in FIG. 4 (second from bottom line), and a Raman spectrum as shown in FIG. 13A. In the Raman spectrum of Polymorph J when compared to Polymorphs A and E (FIGS. 13C and 13D, respectively), the peak patterns are significantly different in the wavenumber around 700 to 740 cm⁻¹ and 1300 to 1340 cm⁻¹.

It is believed that Polymorph J forms under basic conditions (˜pH 9-10). Further, Polymorph J converts to Polymorph A under right conditions as discussed in Example 4.

### Example 2. Preparation of Compound I Polymorph K (Free Base)

In the procedure discussed in Example 1, the wet cake obtained in the Neutralization/Polymorph Formation step was vacuum dried at no more than 65° C. Then the dried Compound I (dried wet cake) was dissolved in DCM/MeOH (3/1, v/v) and purified on silica gel column eluting with DCM/MeOH (3/1, v/v). The fractions containing Compound I was collected and passed through an in-line filter. Then Compound I was precipitated from DCM/MeOH via solvent-swap with MeOH (under vacuum with the jacket temperature of no more than 30C). After the DCM was distilled off, Compound I in MeOH was filtered and the wet cake was washed with methanol. The wet cake was vacuum dried at no more than 65° C. Then the dried Compound I, which existed in Polymorph E, was stirred in MeOH/PPW (3:1) and heated at about 60° C. to about 65° C. for 4 hours. Then the temperature was cooled down to 20° C. to 30° C. and stirred at that temperature for no longer than 1 hour. The contents were filtered and dried to obtain Polymorph K, which was observed to precipitate at neutral pH. In the final heating step, if the mixture is heated for 6 hours instead of 4 hours, Polymorph A was observed.

Polymorph K exhibited an XRPD pattern as shown in FIG. 5 (top line), a DSC thermogram as shown in FIG. 6 (bottom line), a TGA thermogram as shown in FIG. 4 (bottom line), and a Raman spectrum as shown in FIG. 13B. The Raman spectrum of Polymorph K is substantially similar to the Raman spectrum of Polymorph J (FIG. 13A). In the Raman spectrum of Polymorph K when compared to Polymorphs A and E (FIGS. 13C and 13D, respectively), the peak patterns are significantly different in the wavenumber around 700 to 740 cm⁻¹ and 1300 to 1340 cm⁻¹.

It is believed that Polymorph K was transformed from Polymorph E under neutral conditions. Further, Polymorph K converts to Polymorph A (thermodynamically stable form) upon prolonged heating under neutral conditions. Thus, without bound to be any theory, Polymorph K is believe to be a metastable form between Polymorphs E and A.

### Example 3. Solubility of 2-(4-Methyl-[1,4]diazepan-1-yl)-5-oxo-5H-7-thia-1,11b-diaza-benzo[c]fluorene-6-carboxylic acid (5-methyl-pyrazin-2-ylmethyl)-amide (Compound I, free base)

A quick solubility analysis was conducted on Compound I Polymorphs A and J (Table 3). Each sample was suspended in pH 4.5 buffer solution (25 mM sodium acetate, 25° C.) for 10 minutes and then filtered for LC (liquid chromatography) analysis. The concentrations of the test samples saturated at 26 mg/mL-29 mg/mL as summarized in Table 3. Although the pH value in the test sample solutions changed to 5-6, indicating that the concentration of the selected buffer solution may not be suitable for this solubility evaluation, without bound to any theory, it is believed that the solubility of these samples are comparable (i.e., Polymorphs A and J have similar solubility).

TABLE 3
Solubility of Compound I Polymorphs J, A, and mixture of J and A

| | Solubility |
|---|---|
| Compound I Form | (mg/mL) |
| Polymorph A | 29.3 |
| Polymorph J | 28.6 |

### Example 4. Polymorphic Equilibrium Study and Polymorphic Transformation Study for Compound I Polymorph J

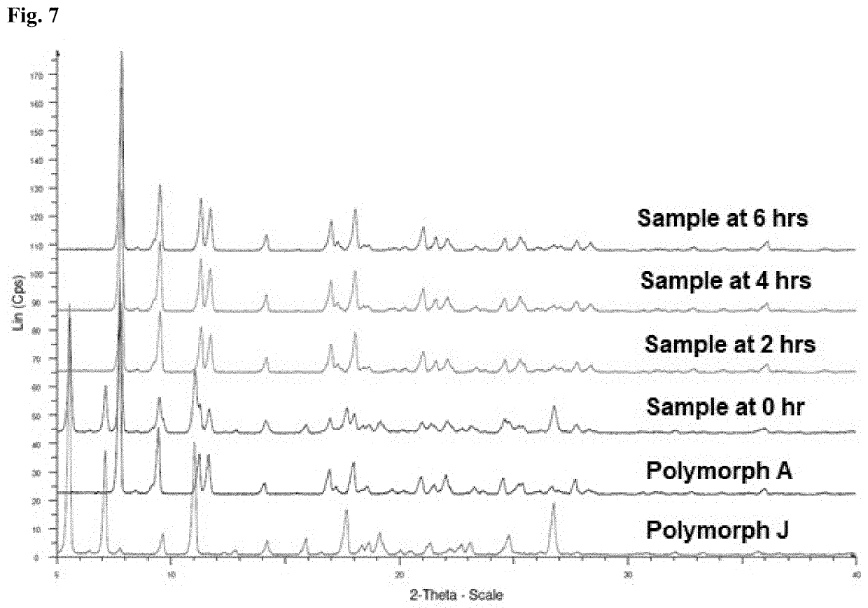

Polymorphic equilibrium studies were performed using Compound I Polymorph A and Compound I Polymorph J. To a 3-neck 250 mL flask equipped with a 3 cm stir bar and N₂ inlet, a 1:1 mixture of Compound I Polymorph A (5 g) and Compound I Polymorph J (5 g, sample shown in FIG. 1), MeOH (112.5 mL, 11.25 vol) and purified process water (PPW, 37.5 mL, 3.75 vol) were charged. The resulting mixture was stirred at room temperature for 10 min, and then heated to 65° C. and sampled at 2 hrs, 4 hrs, and 6 hrs and analyzed by XRPD analysis as shown in FIG. 7. The results indicated that the 1:1 mixture of Polymorphs A and J converted to Polymorph A after heating for 2 hrs at 65° C., demonstrating that Polymorph A is more thermodynamically stable than Polymorph J.

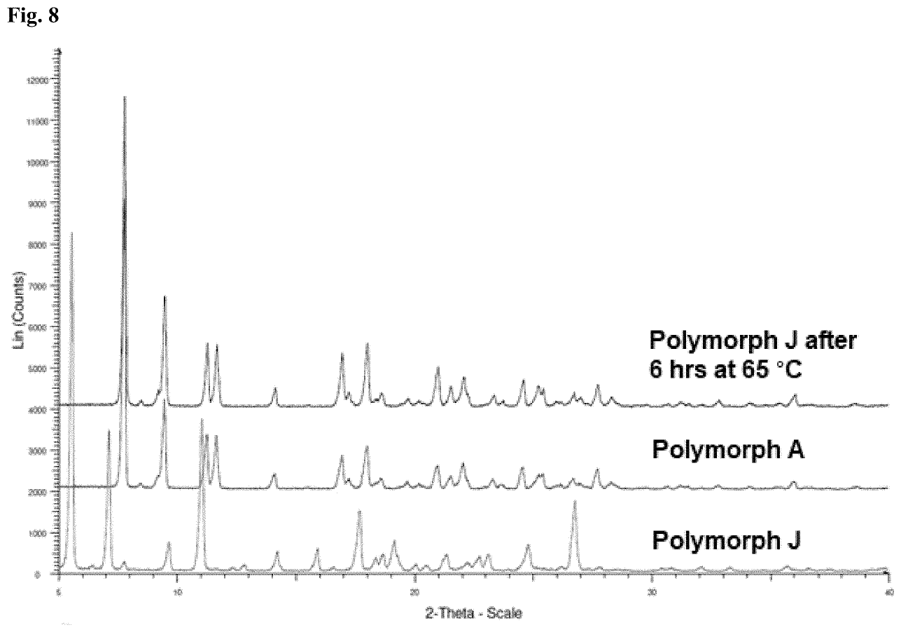

A polymorphic transformation study was also performed. To a 20 mL vial containing a 2 cm stir bar, Compound I Polymorph J (0.5 g, sample shown in FIG. 1), MeOH (5.6 mL, 11.25 vol) and PPW (1.9 mL, 3.75 vol) were charged. The resulting mixture was heated to 65° C. for 6 hrs. Upon completion, the slurry was filtered through a Buchner funnel, and the solid was collected for XRPD analysis as shown in FIG. 8. The result indicated that Polymorph A was indeed obtained after 6 hrs at 65° C. That is, Polymorph J was successfully converted to Polymorph A.

### Example 5. Stability Test of Compound I Polymorph J

As described in Example 1, Polymorph J crystals precipitated out around pH 9-10 during final pH adjustment step to pH 12-13. Polymorph J is then heated to 60° C. to 65° C. in MeOH/PPW (3:1) for approximately 4 hours and maintains its polymorphic form. Thus, substantially pure (polymorphic purity) Polymorph J in MeOH/PPW (3:1) is stable at 65° C. for at least 4 hours.

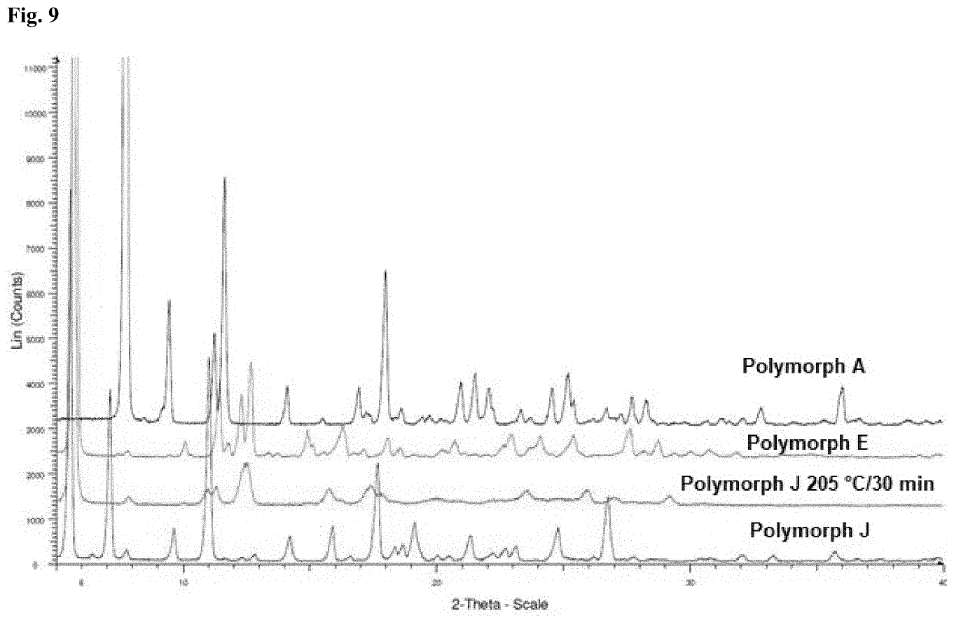

On the other hand, Polymorph J when heated without solvent at 205° C. for 30 minutes appeared to be less crystalline as shown in FIG. 9.

Further, stability of Polymorph J was analyzed in solution having the composition:

| | Quantity per mL |
|---|---|
| Compound I Polymorph J | 30.0 mg |
| Sucrose | 20.0 mg |
| HCl | As needed, for pH adjustment |
| NaOH | As needed, for pH adjustment |
| Water for Injection (WFI) | q.s. |

Sample Preparation: 37.5 kg of WFI was added to the compounding vessel at a temperature in the range of 15 to 30° C. Vigorously sparge WFI with nitrogen for no less than 30 minutes by placing the nitrogen sparging tubing at the bottom of the pressure vessel. Continue sparging in the vessel until dissolved oxygen content was <1 ppm. In a second pressure vessel was added 20.0 kg nitrogen sparged WFI and sucrose and mixed until dissolved while continuing nitrogen sparging, not less than 15 minutes. Nitrogen sparging continued as necessary until dissolved oxygen content was <1 ppm of the sucrose solution. To the sucrose solution, 813.8 g of 2 M HCl solution was slowly added and mixed for no less than 10 minutes after addition was complete. Add Compound I into the sucrose solution vessel and rinse container that contained Compound I with nitrogen sparged WFI. Mix solution until dissolved (no less than 15 min). Add 43.5 mL of 2 M HCl and mix for no less than 5 minutes. If solution is not visually dissolved, add another portion of 43.5 mL of 2M HCl and mix for no less than 5 minutes. Adjust pH, if necessary to 4.4-4.6 with 2M HCl or 1M NaOH solution prepared using nitrogen sparged WFI. Mix solution after each addition of 2M HCl or 1M NaOH. Adjust volume with nitrogen-sparged WFI as necessary. Pull a 10 mL sample to measure pH. If necessary, re-adjust pH to 4.4-4.6 using 2M HCl or 1M NaOH solution prepared using nitrogen sparged WFI. Filtration and filling directly proceeded this step. No material was stored overnight.

Sterilization through 0.22 μM membrane filters: A standard sterile filtration operation was designed to perform sterilization of the compounded bulk solution by membrane filtration through two 0.22 μM hydrophilic polyvinylidene fluoride (PVDF) membranes contained in a polycarbonate housing. The compounded bulk passed through the two sterilizing membranes in series, as is typical in sterile filtration operations, to provide redundant sterilizing capability.

Aseptic filling of the sterile solution: The Compound I sterile solution was filled into 20-cc clean, de-pyrogenated glass vials, with periodic weight checks to assure that the target fill quantity (5.05 g/vial) was maintained, and the vials were semi-stoppered with sterile elastomeric closures to provide the sample for stability analysis.

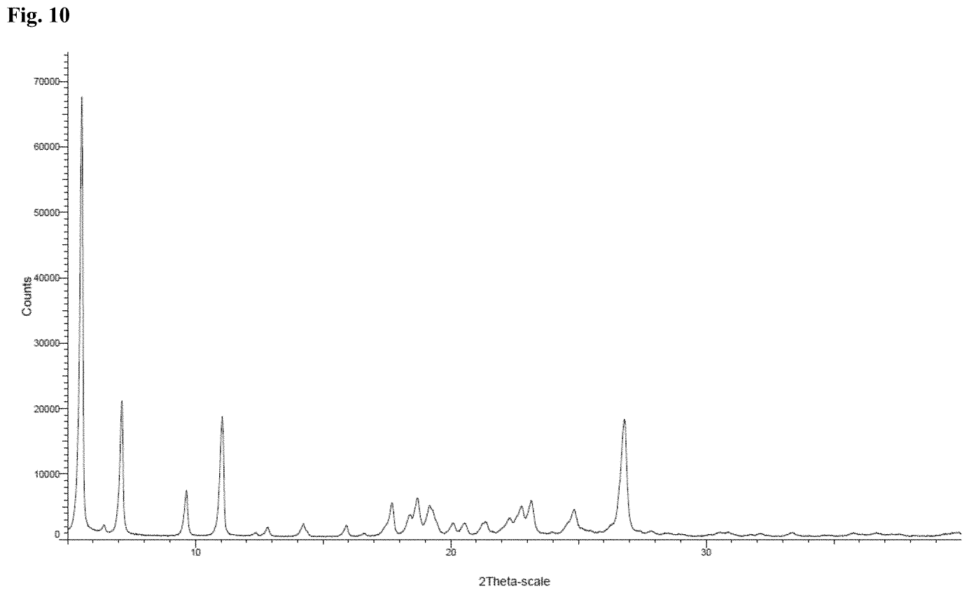

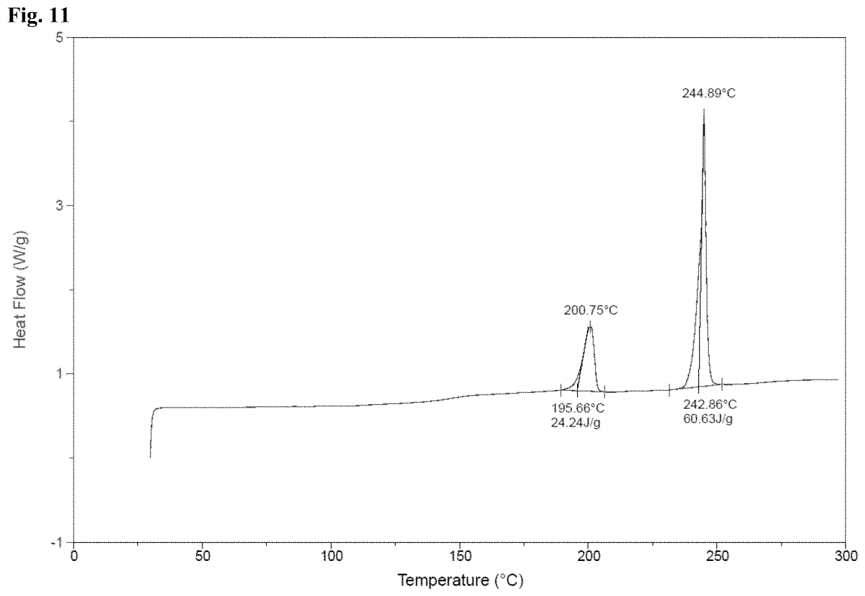

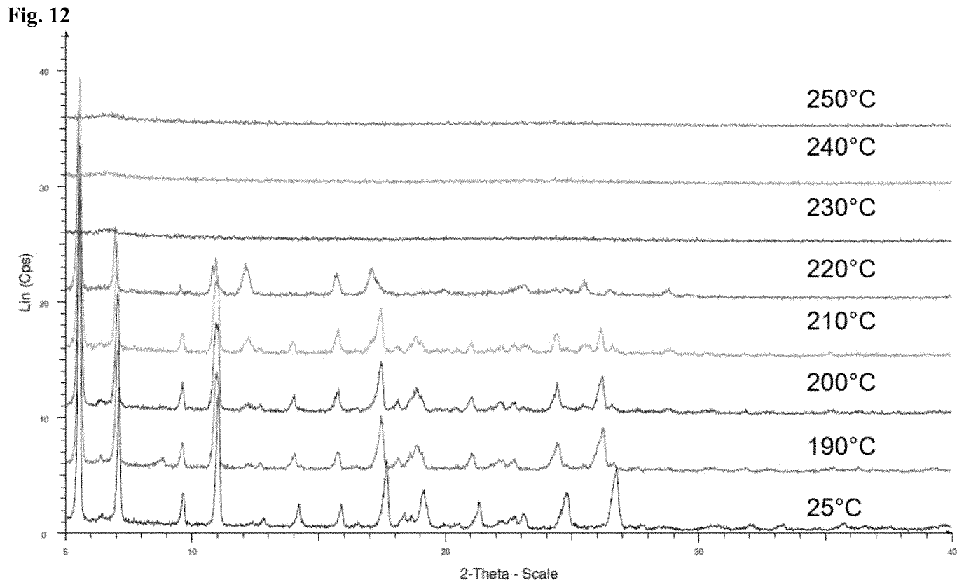

The sample was stored at 25° C. in 60% relative humidity (RH) for 18 months. After 18 months, the sample was analyzed by XRPD and DSC (see FIGS. 10 and 11). XRPD pattern was substantially similar to the XRPD pattern of the initial sample (FIG. 2). The DSC thermogram showed that the second endothermic peak has shifted by about 5° C. when compared to the initial sample (FIG. 3). A VT-XRPD analysis showed that there was a slight rearrangement in the crystal lattice that caused this minor shift (FIG. 12). A solid transition for Polymorph J was observed at 190° C. to 220° C. by VT-XRPD and the disappearance of the diffraction peaks indicated that sample melted at 230° C. The appearance of the sample turned into dark color at the end of the study, which showed that the sample decomposed.

In sum, this study indicates that Polymorph J is fairly stable for at least 18 months at 25° C./60% RH conditions.

### Example 6. Cell Viability Assessment and Cell Proliferation Assessment

The effect of Compound I on cell viability was assessed by Alamar Blue assay of metabolic activity in various cancer cell lines. Table 4 shows Compound I demonstrate broad spectrum antiproliferative activity in multiple cancer cell lines, while being significantly less active in normal cells.

TABLE 4
Compound I EC₅₀ in Cell Viability Assay

| Cell Line | Cancer Type | EC₅₀ (nM) |
|---|---|---|
| EOL-1 | Leukemia | 3 |
| SR | Leukemia | 5 |
| MOLT-3 | Leukemia | 6 |
| MV 4; 11 | Leukemia | 12 |
| SEM | Leukemia | 18 |
| A7 | Melanoma | 23 |
| NCI-H460 | Lung | 38 |
| THP-1 | Leukemia | 47 |
| NCI-H1299 | Lung | 55 |
| A375 | Melanoma | 58 |
| Jurkat | Leukemia | 64 |
| Ramos | Lymphoma | 66 |
| RPMI-8226 | Myeloma | 68 |
| NCI-H520 | Lung | 70 |
| MIA PaCa-2 | Pancreatic | 74 |
| SK-OV-3 | Ovarian | 78 |
| HL60 | Leukemia | 83 |
| MDA-MB-231 | Breast | 83 |
| BT-474 | Breast | 86 |
| COLO-205 | Colon | 96 |
| K562 | Leukemia | 104 |
| Hs 605.T | Breast | 116 |
| ZR-75-1 | Breast | 123 |
| Raji | Lymphoma | 133 |
| SKBr3 | Breast | 134 |
| MDA-MB-453 | Breast | 140 |
| Daudi | Lymphoma | 142 |
| HL60/MX2 | Leukemia | 147 |
| SK-MEL-24 | Melanoma | 147 |
| HCT-116 | Colon | 164 |
| NK92mi | Lymphoma | 165 |
| MDA-MB-468 | Breast | 171 |
| NCI-H2170 | Lung | 194 |
| U2OS | Osteosarcoma | 281 |
| BT-20 | Breast | 335 |
| MCF 7 | Breast | 347 |
| SUM 190PT | IBC* | 583 |
| BxPC-3 | Pancreatic | 664 |
| HT-29 | Colon | 741 |
| SUM 149PT | IBC* | 751 |
| PC-3 | Prostate | 1,100 |
| SK-MES-1 | Lung | 1,260 |
| Hs 578.T | Breast | 1,647 |
| UACC-812 | Breast | 1,830 |
| MDA-MB-361 | Breast | 2,100 |
| T47D | Breast | 2,337 |
| MDA-MB-175-VII | Breast | 2,780 |
| A549 | Lung | 4,900 |
| Saos-2 | Osteosarcoma | 5,000 |
| PANC-1 | Pancreatic | 5,000 |
| LNCaP | Prostate | 5,500 |
| CCD-1058Sk | Normal | 4,710 |
| CCD-1094Sk | Normal | 4,810 |
| CCD-1068Sk | Normal | 5,070 |
| BJ-hTERT | Normal | 5,174 |
| CCD-1096Sk | Normal | 5,260 |

*IBC = Invasive ductal breast carcinoma (inflammatory)

### Example 7. Evaluation of Predictive Biomarkers of Response to Compound I

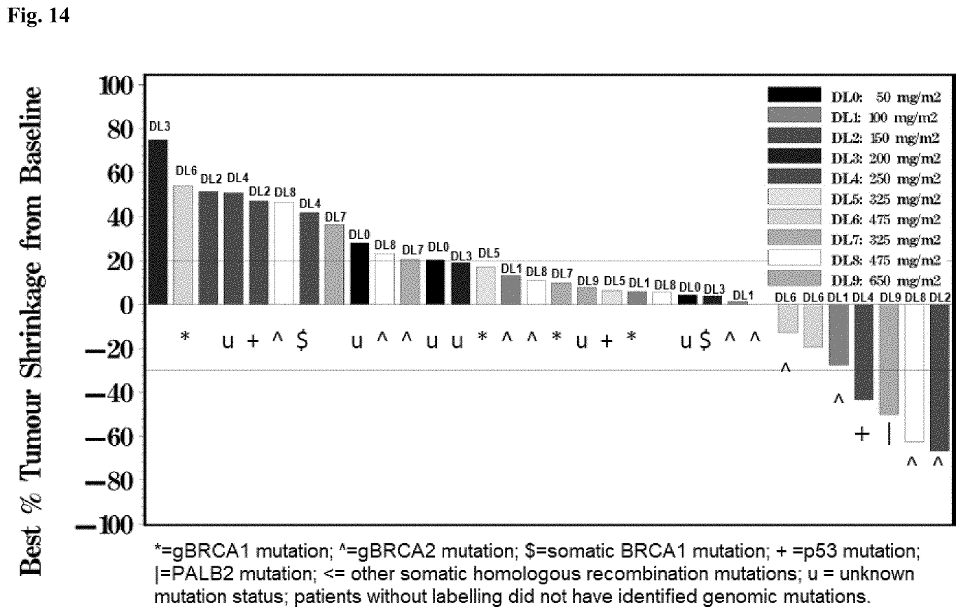

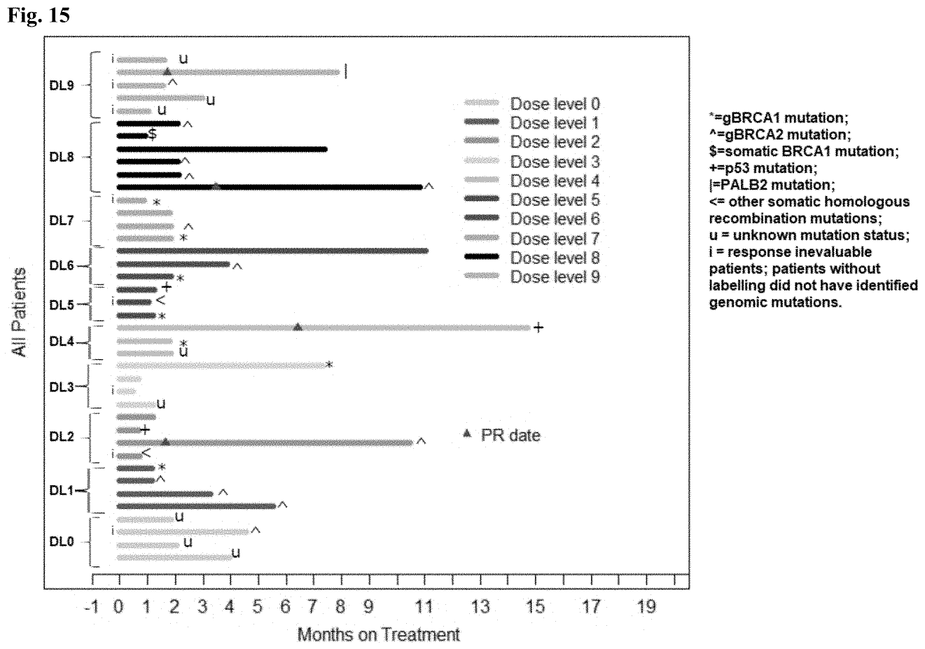

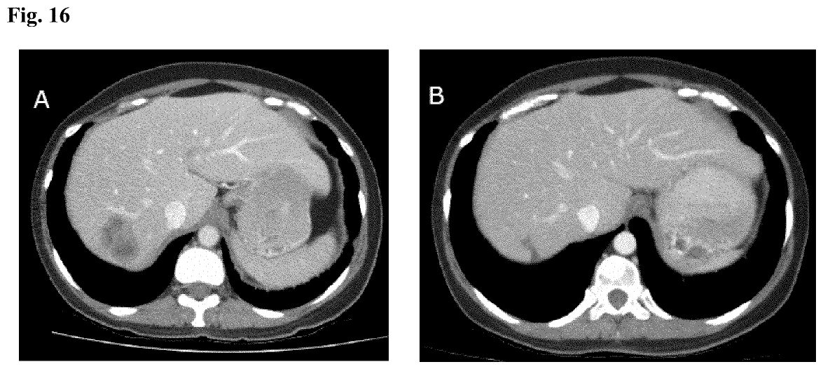

FIG. 14 shows % tumor shrinkage from baseline at each dose level in patients with genetic mutations in gBRCA1, gBRCA2, somatic BRCA, p53, PALB2 or other somatic homologous recombination mutations. Patients with unknown mutation status are labelled “u” in FIG. 14 and patients without labelling did not have identified genomic mutations. The duration on therapy at each dose level for evaluable patients is depicted in FIG. 15.

18 patients were diagnosed with metastatic breast cancer. Of the 18 patients, 10 patients with metastatic breast cancer with BRCA12 germline and relevant somatic mutations, who did not receive prior PARP inhibitor treatments were enrolled in an ongoing study to assess predictability of biomarkers related to breast cancer. This study was conducted to evaluate predictive biomarkers of response to Compound I and to explore the relationship between germline HRD aberrations and outcomes of Compound I treatments.

1 patient (dosed at 650 mg/m²), who harbored a PALB2 mutation and BRCA2 mutation and showed partial response (PR) to Compound I treatment.

This study indicated that patients with BRCA2 mutation responded to Compound I treatment at a dose greater than or equal to 150 mg/m², where tumor shrinkage were observed.

The patents and publications listed herein describe the general skill in the art and are hereby incorporated by reference in their entireties for all purposes and to the same extent as if each was specifically and individually indicated to be incorporated by reference.

In the case of any conflict between a cited reference and this specification, the specification shall control. In describing embodiments of the present application, specific terminology is employed for the sake of clarity. However, the invention is not intended to be limited to the specific terminology so selected. Nothing in this specification should be considered as limiting the scope of the present invention. All examples presented are representative and non-limiting. The above-described embodiments may be modified or varied, without departing from the invention, as appreciated by those skilled in the art in light of the above teachings. It is therefore to be understood that, within the scope of the claims and their equivalents, the invention may be practiced otherwise than as specifically described.

What is claimed:

1. A crystalline form of a Compound I: 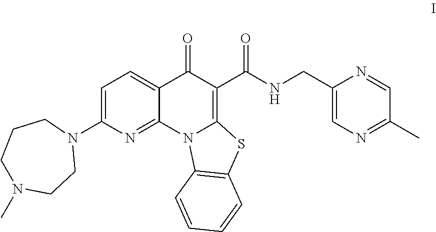 [SMILES: Cc1cnc(CNC(=O)c2c(=O)c3ccc(N4CCCN(C)CC4)nc3n3c2sc2ccccc23)cn1]
wherein the crystalline form is a Polymorph J or Polymorph K.

2. The crystalline form of claim 1, wherein the crystalline form is isolated.

3. The crystalline form of claim 1 or 2, wherein the crystalline form is Polymorph J.

4. The crystalline form of claim 3, which exhibits an X-ray powder diffraction (XRPD) pattern comprising peaks at 5.5±0.5 and 11.0±0.5 degrees two-theta.

5. The crystalline form of claim 3 or 4, which exhibits an XRPD pattern comprising peaks at 7.1±0.5 degrees two-theta.

6. The crystalline form of any one of claims 3-5, which exhibits an XRPD pattern comprising peaks at about 17.7±0.5 and 26.7±0.5 degrees two-theta.

7. The crystalline form of any one of claims 3-6, which exhibits an XRPD pattern substantially similar to FIG. 1 or FIG. 2.

8. The crystalline form of any one of claims 3-7, which exhibits a Differential Scanning Calorimetry (DSC) thermogram having a peak maximum between 200.0° C.±0.5° C. to 202.0±0.5° C.

9. The crystalline form of any one of claims 3-8, which exhibits a Differential Scanning Calorimetry (DSC) thermogram having a peak maximum between 238.0° C.±0.5° C. to 246.0±0.5° C.

10. The crystalline form of any one of claims 3-9, which has a polymorphic purity of about 90% or higher.

11. The crystalline form of any one of claims 3-9, which has a polymorphic purity of about 95% or higher.

12. The crystalline form of any one of claims 3-11, which has a chemical purity of about 95% or higher.

13. The crystalline form of any one of claims 3-11, which has a chemical purity of about 98% or higher.

14. The crystalline form of claim 1 or 2, wherein the crystalline form is Polymorph K.

15. The crystalline form of claim 14, which exhibits an X-ray powder diffraction (XRPD) pattern comprising peaks at 5.2±0.5 and 25.5±0.5 degrees two-theta.

16. The crystalline form of claim 14 or 15, which exhibits an XRPD pattern comprising peaks at 11.4±0.5 degrees two-theta.

17. The crystalline form of any one of claims 14-16, which exhibits an XRPD pattern comprising peaks at 14.7±0.5 and 23.4±0.5 degrees two-theta.

18. The crystalline form of any one of claims 14-17, which exhibits an XRPD pattern substantially similar to FIG. 5.

19. The crystalline form of any one of claims 14-18, which exhibits a Differential Scanning Calorimetry (DSC) thermogram having a peak maximum between 144.0° C.±0.5° C. to 150.0±0.5° C.

20. The crystalline form of any one of claims 14-19, which exhibits a Differential Scanning Calorimetry (DSC) thermogram having a peak maximum between 231.0° C.±0.5° C. to 238.0±0.5° C.

21. The crystalline form of any one of claims 14-20, which exhibits a Differential Scanning Calorimetry (DSC) thermogram having a peak maximum between 242.0° C.±0.5° C. to 250.0±0.5° C.

22. The crystalline form of any one of claims 14-21, which has a polymorphic purity of about 90% or higher.

23. The crystalline form of any one of claims 14-21, which has a polymorphic purity of about 95% or higher.

24. The crystalline form of any one of claims 14-23, which has a chemical purity of about 95% or higher.

25. The crystalline form of any one of claims 14-23, which has a chemical purity of about 98% or higher.

26. A composition comprising a crystalline form of any one of claims 1-25.

27. The composition of claim 26, wherein the composition comprises at least one pharmaceutically acceptable carrier.

28. The composition of claim 27, wherein the composition further comprises one or more additional therapeutically active agent.

29. The composition of claim 28, wherein the additional therapeutically active agent is selected from an alkylating agent, an anti-metabolite, a vinca alkaloid, a taxane, a topoisomerase inhibitor, an anti-tumor antibiotic, a tyrosine kinase inhibitor, an immunosuppressive macrolide, an Akt inhibitor, an HDAC inhibitor an Hsp90 inhibitor, an mTOR inhibitor, a PI3K/mTOR inhibitor, a PI3K inhibitor, a CDK (cyclin-dependent kinase) inhibitor, CHK (checkpoint kinase) inhibitor, or a PARP (poly (DP-ribose)polymerase) inhibitor.

30. The composition of claim 29, wherein the PARP inhibitor is olaparib.

31. The composition of claim 28, wherein the additional therapeutically active agent is selected from an antibody or an antigen-binding portion thereof that disrupts the interaction between Programmed Death-1 (PD-1) and Programmed Death Ligand-1 (PD-L1).

32. The composition of claim 28, wherein the additional therapeutically active agent is selected from an anti-PD-1 antibody, a PD-1 antagonist, an anti-PD-L1 antibody, a siRNA targeting expression of PD-1, a siRNA targeting the expression of PD-L1, and a peptide, fragment, dominant negative form, or soluble form of PD-1 or PD-L1.

33. A method for stabilizing G-quadruplexes (G4s) in a subject, the method comprising administering to the subject a therapeutically effective amount of a crystalline form of any one of claims 1-25 or the composition of any one of claims 26-32 comprising a therapeutically effective amount of the crystalline form.

34. The method of claim 33, wherein the stabilizing G4s is in peripheral blood mononuclear cells.

35. A method for modulating p53 activity in a subject, the method comprising administering to the subject a therapeutically effective amount of a crystalline form of any one of claims 1-25 or the composition of any one of claims 26-32 comprising a therapeutically effective amount of the crystalline form.

36. A method for treating or ameliorating cell proliferation disorder in a subject, the method comprising administering to a subject in need thereof a therapeutically effective amount of a crystalline form of any one of claims 1-25 or the composition of any one of claims 26-32 comprising a therapeutically effective amount of the crystalline form.

37. The method of claim 36, wherein the cell proliferation disorder is cancer.

38. The method of claim 37, wherein the cancer is selected from hematologic malignancy, colorectal cancer, breast cancer, lung cancer, liver cancer, ovarian cancer, cervical cancer, Ewing's sarcoma, pancreatic cancer, cancer of the lymph nodes, colon cancer, prostate cancer, brain cancer, bone cancer, cancer of the head and neck, skin cancer, kidney cancer, osteosarcoma, cancer of the heart, uterine cancer, gastrointestinal malignancies, and carcinomas of the larynx and oral cavity.

39. The method of claim 37, wherein the cancer is breast cancer, ovarian cancer, or pancreatic cancer.

40. The method of claim 38, wherein the hematologic malignancy is selected from leukemia, lymphoma, myeloma, and multiple myeloma.

41. The method of claim 36, wherein the cell proliferation disorder is a solid tumor.

42. The method of claim 36 or 37, wherein the subject has a mutation in a DNA repair gene.

43. The method of claim 42, wherein the DNA repair gene is a gene in the homologous recombination (HR) or non-homologous end joining (NHEJ) dependent deoxyribonucleic acid (DNA) double strand break (DSB) repair pathway.

44. The method of claim 37, wherein the cancer is a BRCA mutant or BRCA-like mutant cancer.

45. The method of claim 37, wherein the cancer is a BRCA mutant cancer.

46. The method of claim 45, wherein the cancer is a BRCA2-mutated cancer.

47. The method of claim 55, wherein the BRCA mutant or BRCA-like mutant cancer is breast cancer, ovarian cancer, pancreatic cancer, or prostate cancer.

48. The method of claim 47, wherein the BRCA mutant cancer is breast cancer, ovarian cancer, pancreatic cancer, or prostate cancer.

49. The method of claim 37, wherein the cancer is BRCA2 deficient or BRCA1 deficient cancer.

50. The method of claim 49, wherein the cancer is BRCA2 deficient cancer.

51. The method of claim 37, wherein the subject has a PALB2 mutation.

52. The method of claim 50, wherein the subject has a PALB2 mutation.
